(19) United States
(12) Patent Application Publication
(10) Pub. No.: US 2024/0274263 A1
(43) Pub. Date: Aug. 15, 2024

(54) SYSTEM AND METHOD FOR GENERATION AND TESTING OF CLINICAL DECISION SUPPORT PROTOCOLS TO ASSIST WITH TARGETED PATIENT INTERVENTIONS

(71) Applicant: Etiometry Inc., Boston, MA (US)

(72) Inventors: Dimitar V. Baronov, Weston, MA (US); Robert Hammond-Oakley, Somerville, MA (US); Evan J. Butler, New Haven, CT (US)

(21) Appl. No.: 18/438,452

(22) Filed: Feb. 10, 2024

Related U.S. Application Data
(63) Continuation-in-part of application No. 17/502,005, filed on Oct. 14, 2021.
Application No. 17/502,005 is a continuation-in-part of application No. 17/402,256, filed on Aug. 13, 2021, now Pat. No. 11,763,947.
(60) Provisional application No. 63/444,875, filed on Feb. 10, 2023.
(60) Provisional application No. 63/444,869, filed on Feb. 10, 2023.
(60) Provisional application No. 63/190,070, filed on May 18, 2021.
(60) Provisional application No. 63/183,979, filed on May 4, 2021.
(60) Provisional application No. 63/180,881, filed on Apr. 28, 2021.
(60) Provisional application No. 63/091,493, filed on Oct. 14, 2020.
(60) Provisional application No. 63/091,427, filed on Oct. 14, 2020.

(51) Int. Cl. G16H 20/40; A61B 5/00; A61B 5/0205; G16H 10/40; G16H 10/60; G16H 15/00; G16H 20/10; G16H 50/30; G16H 50/70

(52) U.S. Cl. CPC G16H 20/40; A61B 5/0205; A61B 5/4842; A61B 5/7282; A61B 5/743; A61B 5/7435; A61B 5/746; G16H 10/40; G16H 10/60; G16H 15/00; G16H 20/10; G16H 50/30; G16H 50/70

(57) ABSTRACT

A system simulates a medical protocol. The system includes an application simulator configured to receive an eligibility rule and compliance rule for a medical protocol. The simulator is further configured to receive collected patient data from a plurality of patients. The collected patient data includes longitudinal patient data relating to a specific parameter collected over a period of time from one or more sensors and/or medical devices. The simulator is further configured to compare the patient data with the eligibility rule to determine when a patient was eligible for the protocol. The simulator is further configured to compare the patient data with the compliance rule to determine a historic dynamic concordance rate for the protocol. The historic dynamic concordance rate for the protocol indicates the rate at which the patient data was consistent with the compliance rule for the protocol over the period of time.

### PRIORITY

This patent application claims the benefit of U.S. Provisional Patent Application No. 63/444,875, filed Feb. 10, 2023, and U.S. Provisional Patent Application No. 63/444,869, filed Feb. 10, 2023, each of which is incorporated by reference herein in its entirety.

This application is also a continuation-in-part application of U.S. patent application Ser. No. 17/502,005, filed Oct. 14, 2021, which is a continuation-in-part of U.S. patent application Ser. No. 17/402,256, filed Aug. 13, 2021, both naming Dimitar V. Baronov, Robert Hammond-Oakley, and Evan J. Butler as inventors, which claims priority to U.S. Provisional Patent Application No. 63/091,493, filed Oct. 14, 2020, U.S. Provisional Patent Application No. 63/091,427, filed Oct. 14, 2020, U.S. Provisional Patent Application No. 63/180,881, filed Apr. 28, 2021, U.S. Provisional Patent Application No. 63/183,979, filed May 4, 2021, and U.S. Provisional Patent Application No. 63/190,070, filed May 18, 2021, each of which is incorporated by reference herein in its entirety.

### FIELD OF THE INVENTION

Illustrative embodiments of the invention generally relate to systems and methods for patient monitoring and, more particularly, illustrative embodiments relate to generating, simulating, and editing a medical protocol.

### BACKGROUND OF THE INVENTION

Practicing medicine is becoming increasingly more complicated due to the introduction of new sensors and treatments. As a result, clinicians are confronted with an avalanche of patient data, which needs to be evaluated and well understood in order to prescribe the optimal treatment from the multitude of available options, while reducing patient risks. One environment where this avalanche of information has become increasingly problematic is the Intensive Care Unit (ICU). There, the experience of the attending physician and the physician's ability to assimilate the available physiologic information have a significant impact on the clinical outcome. Hospitals that do not maintain trained intensivists around the clock experience a 14.4% mortality rate as opposed to a 6.0% rate for fully staffed centers. It is estimated that raising the level of care to that of average trained physicians across all ICUs can save 160,000 lives and $4.3 billion annually. As of 2012, there is a shortage of intensivists, and projections estimate the shortage will only worsen, reaching a level of 35% by 2020.

The value of experience in critical care can be explained by the fact that clinical data in the ICU is delivered at a rate far greater than even the most talented physician can absorb, and studies have shown that errors are six times more likely under conditions of information overload and eleven time more likely with an acute time shortage. Moreover, treatment decisions in the ICU heavily rely on clinical signs that are not directly measurable, but are inferred from other physiologic information. Thus, clinician expertise and background play a more significant role in the minute to minute decision making process.

### SUMMARY OF VARIOUS EMBODIMENTS

In accordance with one embodiment of the invention a computer-implemented method generates a clinical decision support application. The method configures a protocol eligibility condition by selecting one or more eligibility condition objects from an available list within a configuration graphical user interface. Each eligibility condition object is associated with at least one corresponding condition type, and each type is configurable with specific parameters. Protocol compliance conditions are configured by selecting one or more compliance condition objects from an available list within the configuration graphical user interface. Each compliance condition object is related to a patient parameter. Thresholds within which compliance is achieved for each patient related parameter are selected. The patient related parameters are arranged by an order in which to display them in an active graphical user interface. A list of notification conditions are configured by selecting from a list of specific notification types under which a user of the clinical decision support application will be notified, and each notification type is associated with at least one of a plurality of users. The method then generates an executable clinical decision support application code.

In various embodiments, an interface may be provided that is configured to receive the conditions from the graphical user interface and generate the executable clinical decision support application code as a function of the received conditions.

Among other things, the plurality of eligibility condition objects includes a rate based object, a measurement based object, and an event based object. Similarly, the plurality of compliance condition objects may include a rate based object, a measurement based object, and an event based object.

In some embodiments, a second protocol condition object is selected from the configuration graphical user interface. The second protocol condition object is different from the first protocol condition object. The second selected protocol condition object is displayed with the first selected protocol condition object in the protocol sequence window of the graphical user interface. Furthermore, a second specific parameter window related to the corresponding parameter requirement for the second selected object may be displayed in the sequence window. A specific parameter related to the corresponding parameter requirement may be input within the second specific parameter window. A clinical protocol having conditions as a function of the received specific parameters for the first and the second selected protocol condition types may be generated. In some embodiments, the condition is an eligibility condition or a compliance condition for the protocol. Some embodiments select a plurality protocol condition objects from the graphical user interface. The plurality of protocol condition objects are rearrangeable in the protocol sequence window, and the plurality of protocol condition objects are logically linkable using and/or statements.

The generated protocol may be displayed in an active user interface.

In accordance with another embodiment, a method creates protocols. The method selects a protocol condition object from a graphical user interface displaying a plurality of different protocol condition objects. Each object is associated with at least one corresponding parameter requirement. The selected protocol condition object is displayed in a protocol sequence window of the graphical user interface. A specific parameter window related to the corresponding parameter requirement for the selected object may be displayed. A specific parameter related to the corresponding parameter requirement within the specific parameter window is received. A clinical protocol having a condition as a function of the received specific parameter for the selected protocol condition type is generated.

In various embodiments, a plurality protocol condition objects are selected from the graphical user interface. The plurality of protocol condition objects may be rearrangeable in the protocol sequence window. A rule may include the plurality of protocol condition objects e logically linked using and/or statements. The generated protocol may be displayed in a patient display. Patient data may be collected relating to the generated protocol and displaying data relevant to the generated protocol in the patient display.

In some embodiments, the eligibility condition is a parent based condition. In some embodiments, the eligibility condition may be a function of an internal state variable. In some embodiments, the eligibility condition may be an event based condition.

In accordance with another embodiment, a system creates a medical protocol. The system includes a conditions library containing graphically selectable eligibility objects and graphically selectable compliance objects relating to a medical protocol. The system also includes a graphic compiler generating a user interface. The user interface is configured to display the graphically selectable eligibility objects and graphically selectable compliance objects. The user interface is further configured to display a sequence window in which the graphically selectable eligibility objects may be moved to define an eligibility rule for the protocol. The graphically selectable compliance objects may be moved in the sequence window to define a compliance rule for the protocol.

In various embodiments, the system includes a reporting module configured to provide reports to medical practitioners. The reports may be configured using the graphical compiler. The graphical reporting objects may relate to, among other things, adoption by the medical practitioner. Examples of graphical objects for reporting include counting clicks by medical staff in the interface, counting interactions with the interface by medical staff, average patient compliance, time from eligibility to protocol start, ICU length of stay, ventilation time, extubation failure rate, readmission rate, and/or patient outcomes relative to dynamic concordance of the patient.

In various embodiments, the system also includes an event detector configured to parse the patient data to automatically detect a patient event. Among other things, the patient event may include extubation, reintubation, length of extubation, extubation failure detection, cardiac arrest detection, vasoactive weaning detection, and/or acute kidney injury detection.

In accordance with an embodiment, a method simulates a medical protocol. The method receives an eligibility rule and a compliance rule for a medical protocol. Each of the rules has one or more conditions. Collected patient data from a plurality of patients is received. The collected patient data includes longitudinal patient data relating to a specific parameter collected over a period of time from one or more sensors and/or medical devices. The method determines when a patient was eligible for the protocol as a function of the eligibility rule and the collected patient data. The method determines a historic dynamic concordance rate for the protocol. The historic dynamic concordance rate for the protocol indicates the rate at which the patient data was consistent with the compliance rule for the protocol over the period of time.

In various embodiments, the collected patient data includes data from the EHR. Additionally, the collected patient data may include patient data from a newly deployed medical device. Accordingly, a protocol may be generated that includes one or more conditions relating to the patient data from the newly deployed medical device. The protocol may then be adjusted as a function of the patient data from the newly deployed medical device.

Among other things, the method may report patient outcomes as a function of high compliance with the protocol relative to low compliance with the protocol. If patient outcomes for high compliance are undesirable, the protocol may be adjusted.

The eligibility rule and/or compliance rule for the protocol may be adjusted to define an adjusted protocol. The method may determine patient outcomes as a function of high compliance with the protocol relative to low compliance with the adjusted protocol. The process may be repeated until outcomes for high compliance are desirable.

The method may receive notification rule associated with the protocol. Using the notification rule, a simulated number of notifications over the period of time may be determined. The notification rule may be adjusted to reduce the simulated number of notifications.

In various embodiments, the protocol eligibility rule and compliance rule are generated using the graphic compiler. The protocol eligibility rule and/or compliance rule may be adjusted using the graphical compiler (e.g., after receiving feedback regarding patient outcomes as a function of high compliance with the protocol). The protocol eligibility rule and/or compliance rule may be adjusted using the graphical compiler after receiving feedback regarding a number of notifications generated by the protocol.

In accordance with another embodiment, a system simulates a medical protocol. The system includes an application simulator configured to receive an eligibility rule and compliance rule for a medical protocol. The simulator is further configured to receive collected patient data from a plurality of patients. The collected patient data includes longitudinal patient data relating to a specific parameter collected over a period of time from one or more sensors and/or medical devices. The simulator is further configured to compare the patient data with the eligibility rule to determine when a patient was eligible for the protocol. The simulator is further configured to compare the patient data with the compliance rule to determine a historic dynamic concordance rate for the protocol. The historic dynamic concordance rate for the protocol indicates the rate at which the patient data was consistent with the compliance rule for the protocol over the period of time.

Various embodiments include a database including the eligibility rule and the compliance rule. A graphic compiler may be configured to graphically generate the eligibility rule and/or the compliance rule. The application simulator may configured to receive notification rule associated with the protocol, and to determine the number of notifications as a function of the protocol and the patient data.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a clinical patient environment in accordance with illustrative embodiments of the invention.

FIG. 2 schematically shows a generic example of a medical protocol having an eligibility rule, a compliance rule, a protocol trigger, a subscription rule, and a notification rule in accordance with illustrative embodiments.

FIG. 3 schematically shows an active interface for viewing patient protocol information in accordance with illustrative embodiments.

FIG. 4A schematically shows details of a system in accordance with illustrative embodiments of the invention.

FIG. 4B schematically shows details of a sub-system of the system of FIG. 4A.

FIG. 4C schematically shows details of a sub-system of the system of FIG. 4A.

FIG. 5 shows a process of simulating a clinical protocol in accordance with illustrative embodiments of the invention.

FIG. 6 shows a process of generating the clinical protocol using the graphic compiler in accordance with illustrative embodiments of the invention.

FIGS. 7-9 schematically show generation of an eligibility rule using the user interface of the graphical compiler in accordance with illustrative embodiments of the invention.

FIG. 10 schematically shows selection of display options using the user interface of the graphical compiler in accordance with illustrative embodiments.

FIG. 11 schematically shows the selected display options from FIG. 10 in the user interface of FIG. 3.

FIG. 12 schematically shows generation of the compliance rule using the user interface of the graphical compiler in accordance with illustrative embodiments of the invention.

FIG. 13 schematically shows selection of display options using the configuration user interface of the graphical compiler in accordance with illustrative embodiments.

FIG. 14 schematically shows the active user interface configured with the selected compliance visualizations shown in FIG. 13 in accordance with illustrative embodiments.

FIGS. 15-17 schematically show the user interface for configuring the notification rule in accordance with illustrative embodiments.

FIG. 18 schematically shows the configuration user interface for the reporting module in accordance with illustrative embodiments.

FIG. 19 schematically shows the configuration user interface of FIG. 18 after selection of the graphical object in accordance with illustrative embodiments.

FIG. 20 schematically shows a report generated based on the selections in FIGS. 18-19 in accordance with illustrative embodiments.

FIG. 21 schematically shows generation of reporting conditions using the user interface of the graphical compiler in accordance with illustrative embodiments of the invention.

FIG. 22 schematically shows a report generated based on the selections in the adherence sub-menu interface in accordance with illustrative embodiments.

FIG. 23A schematically shows generation of reporting conditions using the user interface of the graphical compiler in accordance with illustrative embodiments of the invention.

FIG. 23B shows the interface of FIG. 23A after the user has selected graphical objects in accordance with illustrative embodiments.

FIG. 24 schematically shows a report generated based on the selected graphical objects from FIG. 23B in accordance with illustrative embodiments.

FIG. 25 schematically shows the user interface of the graphical compiler in accordance with illustrative embodiments of the invention FIG. 26A schematically shows the extubation failure rate report for a first protocol in accordance with illustrative embodiments.

FIG. 26B schematically shows the extubation failure rate report for a second protocol in accordance with illustrative embodiments.

FIGS. 27-28 schematically show the user interface for configuring reports associated with the protocol in accordance with illustrative embodiments.

FIGS. 29-32 schematically shows views of data collection that may be used by the analyzer to automatically determine clinical events and/or outcomes in accordance with illustrative embodiments.

FIG. 33A shows a process of creating a best practice alert in accordance with illustrative embodiments.

FIG. 33B schematically shows a user interface of the electronic health record in accordance with illustrative embodiments.

FIG. 34 shows a process of generating and optimizing a protocol for newly deployed medical device in accordance with illustrative embodiments.

FIG. 35 schematically shows an example of a protocol using a newly deployed medical device in accordance with illustrative embodiments.

### DESCRIPTION OF ILLUSTRATIVE EMBODIMENTS

In illustrative embodiments, a protocol compiler allows a medical practitioner to generate and/or modify a medical protocol within a graphical user interface. Among other things, the graphical user interface provides selectable protocol eligibility conditions and/or selectable protocol compliance conditions (generically referred to as a “protocol condition”). In particular, one or more selectable protocol condition objects are displayed within the interface. One or more selected protocol condition objects may be used to generate the medical protocol rules (e.g., by combining using logical operators). Depending on the type of condition object, the medical practitioner may enter values for specific parameters within a parameter window in the user interface. Illustrative embodiments provide the parameter window over, or adjacent to, one or more selected model objects. The window may display one or more properties associated with each of the selected protocol condition objects. Additionally, the properties may be edited in the window itself and propagated throughout the entire system.

Furthermore, in illustrative embodiments a protocol simulator allows the medical practitioner to test the performance of the protocol using real historic patient data. The simulator simulates the protocol (e.g., the protocol generated using the compiler or traditional coding methods) and provides outcome performance characteristics for patients (e.g., based on their overall compliance rate with the protocol). The performance characteristics allow for rapid iteration of the protocol based on desired patient and/or clinician performance. For example, if patient performance from the simulation is relatively poor despite high compliance, then the protocol (or specific parameters thereof) may be iterated (e.g., using the compiler) until desired patient performance corresponds to high patient compliance. Additionally, if patient performance is acceptable above a certain compliance threshold, then the medical practitioner can take focus hospital resources to bringing all patients within the desired threshold for compliance.

Additionally, illustrative embodiments enable performance monitoring of medical staff that are responsible for overseeing and executing particular protocols and subsequent interventions. The performance of the medical staff may be output into custom reports. The reporting conditions may be established using the graphical user interface. To make the reporting possible, the system includes a library of functions that analyze the data and extract outcomes from the data. (e.g., determines that a patient failed extubation by analyzing ventilator operation data).

Furthermore, various embodiments enable optimization of protocols particularly as they relate to newly deployed medical devices. Optimization insights may be determined by iterating on multiple versions of protocols with different conditions until a desirable protocol is generated. Details of illustrative embodiments are discussed below.

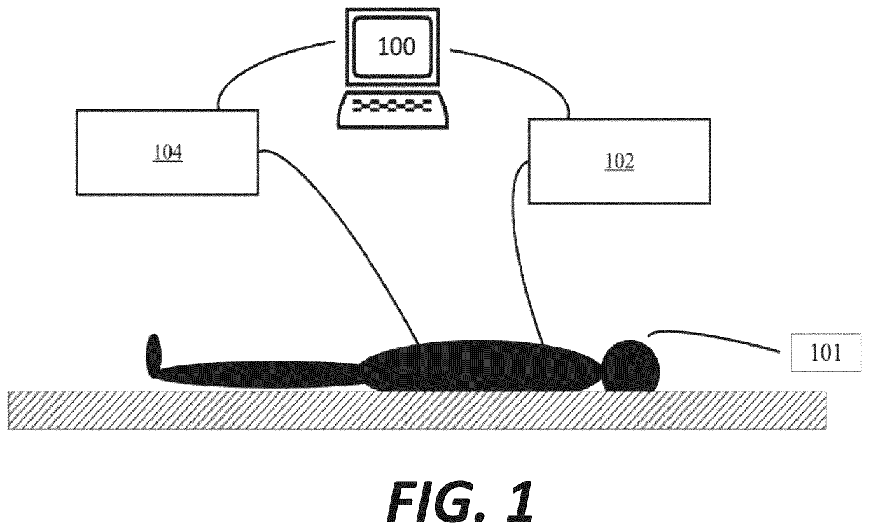

FIG. 1 schematically shows a clinical patient environment in accordance with illustrative embodiments of the invention. In various embodiments, the medical protocols generated using the graphical compiler may be active in the clinical patient environment of FIG. 1. Additionally, or alternatively, the protocol may be simulated using historic data gathered from the clinical patient environment shown herein. In various embodiments, the clinical patient environment may be a specific clinical environment (e.g., a neonatal ICU).

The environment includes sensors 102 (also referred to as monitors 102) for providing patient data to health providers, such as physicians, nurses, or other medical care providers. To that end, a patient 101 may be coupled to one or more physiological sensors 102 or bedside monitors 102 that may monitor various physiological parameters of the patient. It should be noted that a patient 101 may be a human, or not human (a non-human being, e.g., a veterinary patient such as a dog).

The sensors 102 may include, but are not limited to, a blood oximeter, a blood pressure measurement device, a pulse measurement device, a glucose measuring device, one or more analyte measuring devices, an electrocardiogram recording device, amongst others. In addition, the patient 101 may be administered routine exams and tests and the data may be stored in an electronic health record (EHR). The electronic health record may include but is not limited to stored information such as hemoglobin, arterial and venous oxygen content, lactic acid, weight, age, sex, ICD-10 code, capillary refill time, subjective clinician observations, patient self-evaluations, prescribed medications, medications regiments, genetics, test results, allergies, etc.

In addition, the patient 101 may be coupled to one or more treatment devices 104 that are configured to administer treatments to the patient 101. In some embodiments, one or more treatment devices 104 may be controlled by a system as disclosed herein, for example in response to output defining a patient state or medical condition from a trajectory interpreter module. In various embodiments, the treatment devices 104 may include extracorporeal membrane oxygenator, mechanical ventilator, medication infusion pumps, implantable ventricular assist devices, etc.

Illustrative embodiments provide real-time automatic determination of whether the patient 101 is eligible for a course of action of the medical protocol (referred to generally as being eligible for the protocol) based at least partially on data acquired directly from the sensors and peripheral devices. The real-time determination is advantageous over prior art methods that require the medical practitioner to review disparate patient data reports and analyze the data to determine whether the patient 101 is eligible for the protocol. Additionally, in the prior art the medical practitioner generally checks patient eligibility sporadically, leading to sub-optimal clinical outcomes in cases where optimal medical treatment for the patient 101 requires early initiation of the protocol. In a similar manner, sporadic eligibility checks fail to account for the dynamic concordance of the patient's 101 eligibility because the measurements are taken at a static moment in time.

In various embodiments, the system 100 may track how long a patient variable has been in compliance with a set eligibility criteria, advantageously improving medical protocol technology by enabling new protocols that have a longitudinal (e.g., sustained time basis) criteria, as opposed to protocols that use the most-recent static data points. Accordingly, illustrative embodiments track dynamic concordance, or the rate at which patient physiological data is consistent with the requirements of a protocol after the protocol is initiated. This advantageously allows medical practitioners to visualize the patient data as a percentage compliance per time duration (e.g., 60% dynamic concordance in the 3 hours since protocol began). Furthermore, tracking eligibility time advantageously allows medical practitioners to prioritize patients who have been eligible for a long time, thus mitigating any risk emanating from the fact that they have not been getting the prescribed protocol treatment for prolonged duration.

The dynamic concordance calculation based on longitudinal measurements of patient variables also supports using the metric as an actionable therapeutic target and consequently administering treatment targeted at increasing the concordance rate of the patient. Furthermore, the inventors have preliminary data indicating that patients achieving a desired dynamic concordance rate may experience better outcomes relative to patients that have lower concordance rate.

By providing continuous real-time assessment of whether the patient trajectory is progressing according to a protocol specified pathway, the dynamic concordance affords more focused and timely intervention to keep the patient within the most advantageous course, which is a significant improvement relative to what is possible with current technology. The longer time the patient spends within the desired trajectory as measured by the concordance rate the higher is the likelihood that the patient will not experience complications and achieve the optimal possible clinical outcome. Conversely, the less time the patient spends within the desired trajectory as measured by the concordance rate the higher the likelihood that the patient will experience complications and not achieve the optimal clinical outcome.

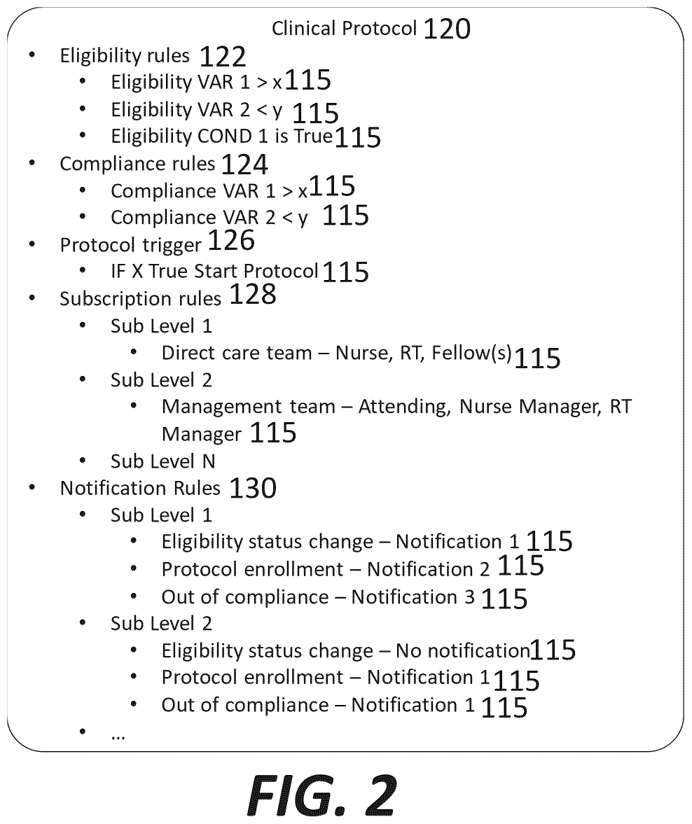

FIG. 2 schematically shows a generic example of a medical protocol 120 having an eligibility rule 122, a compliance rule 124, a protocol trigger 126, a subscription rule 128, and a notification rule 130 in accordance with illustrative embodiments. Each of the aforementioned rules/triggers may have one or more conditions 115. Additionally, in some embodiments, the medical protocol 120 may not include the subscription rule 128, the notification rule 130, the compliance rule 124 and/or the protocol trigger 126.

The eligibility rule 122 may include one or more conditions 115 that must be satisfied (e.g., true or false) for the patient 101 to be eligible for the protocol 120. An eligibility module 110 may compare data in the database 105 and/or EHR with the rule 122 to determine whether the rule 122 is met. Additionally, or alternatively, the eligibility rule 122 may include one or more parametric conditions 115 that must be satisfied (e.g., a certain variable is greater than a particular value). The eligibility module 110 may receive patient data from the device interface 106 to determine whether the patient 101 is eligible (e.g., the conditions 115 of the rule 122 are satisfied). In some embodiments, the eligibility rule 122 of one protocol may be dependent upon the compliance of another protocol (referred to as parent and child protocols), which are described in U.S. patent application Ser. No. 17/502,005, incorporated herein by reference in its entirety.

In a similar manner, the compliance rule 124 may include one or more conditions 115 that must be satisfied (e.g., true or false) for the patient 101 to be in compliance with the protocol 120. The concordance module 110 may compare data in the database 105 and/or the EHR 103 with the rule 122 to determine whether the rule 122 is met (i.e., whether each condition of the rule is met). Although the EHR 103 and the database 105 are shown as separate components, it should be understood that the database 105 may interface with the EHR 103 to extract relevant patient information and store data from the EHR 103 in the database. Thus, illustrative embodiments may make determinations described herein by communicating with the EHR 103, the database 105, or both.

Additionally, or alternatively, the compliance rules 124 may include a variety of parametric conditions that must be satisfied (e.g., a certain variable is greater than a particular value). The system 100 may receive patient data from the device interface 106 to determine whether the patient is in compliance with the protocol 120 (e.g., the rule 124 is satisfied).

Furthermore, in some embodiments the protocol 120 may define a protocol trigger rule 126 that includes one or more conditions 115 that automatically trigger the course of action to begin when satisfied. In some embodiments, the conditions 115 may include a dynamic concordance rate of the given protocol or of a parent protocol. The protocol trigger module 116 may detect when these conditions 115 are satisfied, and may communicate with and/or control one or more medical devices 104 to begin the course of action.

In various embodiments, the protocol 120 may further include a subscription rule 128 and notification rule 130. The subscription rule 128 assign different subscription levels to different medical practitioners. For example, the direct care team may be subscription level 1, whereas the management team by may be subscription level 2. The subscription rule 128 may be used in the notification rule 130. Accordingly, different subscriptions may receive different notifications and/or notifications for different reasons.

Any of the above rules may include conditions 115 based on the probability of a patient being in a physiological state (e.g., in an abnormal physiological state). Various embodiments may use an internal state variable (ISV) (e.g., a hidden internal state variable) as one or more conditions 115. To that end, various embodiments may use probability density functions (PDF) of internal state variables (ISVs) generated by the risk engine 1000 as one or more conditions 115. Additionally, clinical events/outcomes detected by the event detector may be used as one or more conditions 115.

As used herein, the term “rules” (e.g., as used to refer to subscription rules 128, notification rules 130, eligibility rules 122, compliance rules 124) is intended to include a single rule having a single condition 115. For convenience, a rule having multiple conditions may be referred to in the plural, as “rules.” However, the terms are used interchangeably. Illustrative embodiments using the term “rules” do not necessarily require a plurality of conditions. It should be understood that both the singular “rule” or the plural “rules” are intended to include one or more conditions

In a given clinical environment, one or more patients 101 may be on a medical protocol 120 (e.g., the same or different protocols). The sensors 102 of FIG. 1 are collecting data related to the protocol 120 (e.g., a generic example of a protocol shown in FIG. 2). To that end, a patient protocol eligibility and compliance system, generally referred to herein as system 100, may be configured to receive patient related information, including real-time information from the sensors 102, EHR patient information from the electronic health record, information from the treatment devices 104, such as ventilatory settings, infusion rates, types of medications, and other patient related information, which may include the patient's medical history, previous treatment plans, results from previous and present lab work, allergy information, predispositions to various conditions, and any other information that may be deemed relevant to make.

The system 100 provides real-time protocol eligibility updates. Additionally, illustrative embodiments may determine the extent of time that the patient 101 is eligible for a protocol. Accordingly, medical practitioners may have quantifiable standards by which medical practitioner performance is judged (e.g., time from eligibility to protocol initiation, protocol compliance rate, etc.).

To that end, the system 100 may include a display (e.g., coupled to the system 100). In various embodiments, the display may be an interactive display that provides easy visualization of patient performance with respect to eligibility and/or compliance with a particular protocol. In various embodiments, the display provides a listing of all patients under the care of the medical practitioner, or all patients in a particular unit, and further provides protocol status details for each patient. For example, illustrative embodiments provide visual indications of whether the patient is eligible for a course of action associated with the protocol and the amount of time the patient has been eligible. If the patient is on a protocol, illustrative embodiments provide visual indications of how long the patient has been on the protocol and how compliant the patient has been with the protocol. Some embodiments may provide a visual indication of the compliance rate per individual parameter of the protocol for each patient. After selecting the visual indication, a display shows the patient performance variables that are relative to the particular protocol. In various embodiments, this display may be accessed via the EHR 103 interface.

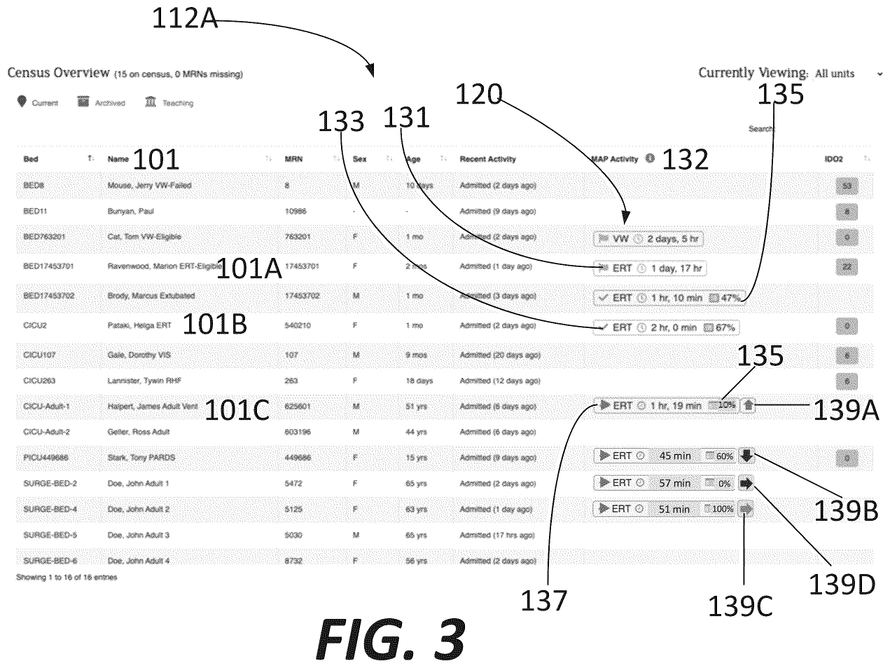

FIG. 3 schematically shows an active interface 112A for viewing patient protocol 120 information in accordance with illustrative embodiments. The patient's 101 medical event history, as well as ongoing protocol, completed protocol, and/or protocol eligibility history can be viewed by the medical practitioner in the interface 112A. Among other things, the interface may show the patient bed number, name, status, health record number, sex, age, recent activity, map activity 132, and/or other parameter values.

The interface 112 displays one or more patients 101 that are eligible for an eligible protocol and/or one or more patients 101 that are enrolled in a protocol. Illustrative embodiments provide a single user interface 112 configured to display patient information relating to a plurality of patients 101. The plurality of patients 101 can be all of the patients 101 on a particular hospital floor, in a particular hospital unit, associated with a particular protocol 120, and/or under the care of a particular medical practitioner. In the same user interface 112, the protocol activity 132 for each patient 101 is shown and described. For example, patient 101A is shown as being eligible (e.g., eligibility icon 131) for an extubation readiness trial (ERT) protocol 120 and the eligibility time (e.g., 1 day and 17 hours for patient 101A). Patient 101B is shown as having completed an ERT protocol 120 (e.g., completed icon 133) and the total time of the completed protocol (i.e., 2 hours and 0 minutes for patient 101B). Patient 101C is shown as currently being enrolled/on-going (e.g., enrollment icon 137) in an ERT protocol 120, and the total time the patient 101C has been on the protocol (e.g., 1 hour and 19 minutes for Patient 101C).

The interface 112 may also show the calculated dynamic concordance rate 135. As shown in FIG. 3, optionally illustrative embodiments may display the amount of time the patient 101 has been or was on the protocol 120, as well as a dynamic concordance rate 135.

In various embodiments, the dynamic concordance rate may be updated in real time, or as new data is received from the sensors and/or medical devices. Some embodiments may update the dynamic concordance rate 135 on a pre-defined schedule (e.g., every 30 seconds, every minute, etc.). The dynamic concordance rate may be displayed as it is updated. The system 100 may determine in which direction the dynamic concordance rate is trending. In illustrative embodiments the trending information may be displayed on the user interface. For example, a dynamic concordance indicator icon 139 may indicate whether the dynamic concordance rate is trending up (e.g., green arrow icon 139A), trending down (e.g., red arrow icon 139B), or is steady within a pre-defined timeframe (e.g., horizontal green arrow 139C if the overall concordance is above a threshold or horizontal red arrow 139D if the overall concordance is below the threshold).

The system 100 may visually indicate completed protocols with a check mark on the user interface 116. These visualization enable simplify transfer of clinical care from one medical practitioner to another, and therefore, lead to better patient outcomes. For example, extubation decisions are usually made by the care team during morning rounds based on extubation readiness trials completed during the previous shift. The rounding team can review which patients have had completed ERTs and then make decision to extubate all patients with high overall concordance. The longitudinal data for patients with lower concordance can be reviewed in more detail to determine reason for noncompliance and assess whether this reason indicates unacceptable risk for extubation failure. Furthermore, in practice, by the time the protocol 120 is complete, it is possible that the medical practitioner that was in charge of the patient may have changed shifts. Illustrative embodiments provide visualizations that simplify prioritization for medical care of patients for subsequent clinicians.

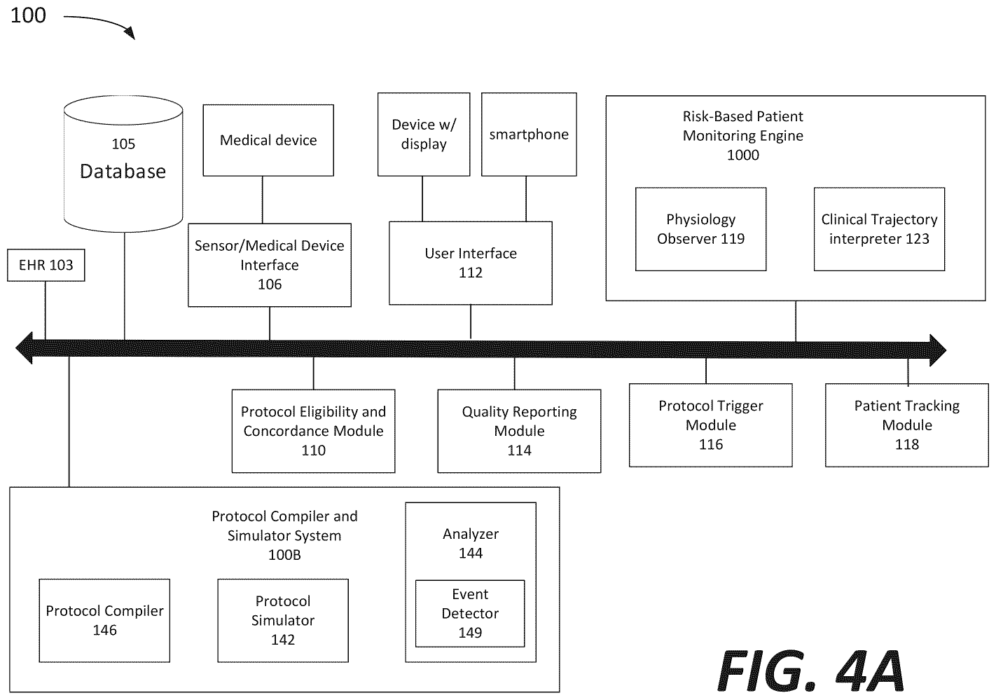

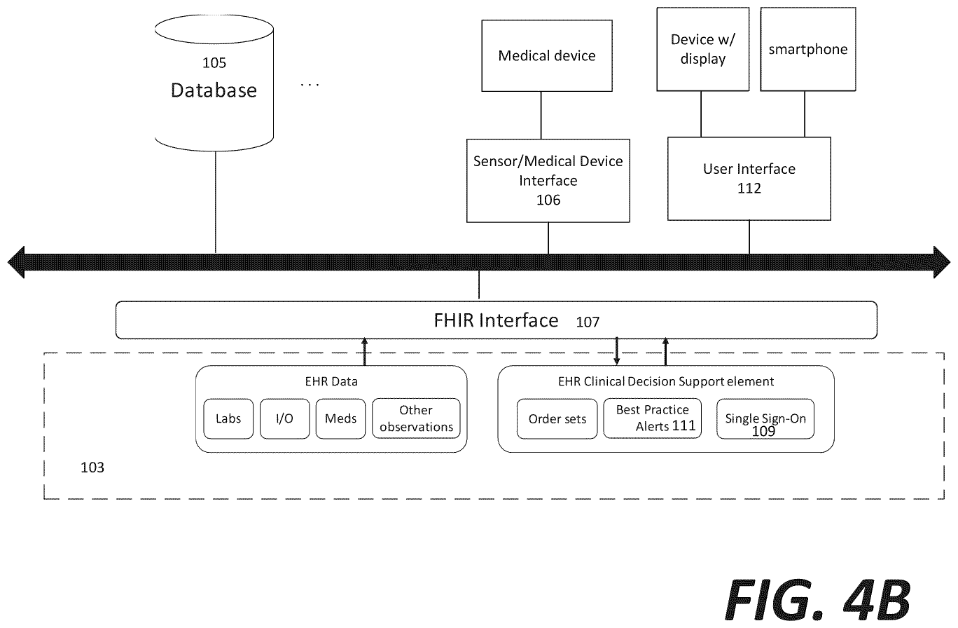

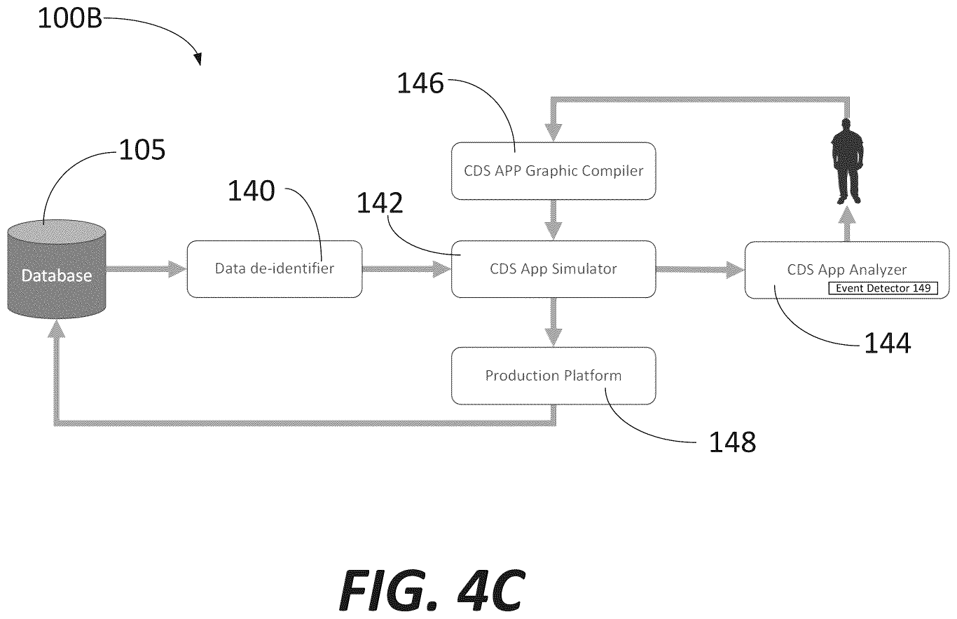

FIG. 4A schematically shows details of the system 100 in accordance with illustrative embodiments of the invention. The system 100 includes a sensor/medical device interface 106 configured to communicate with the one or more sensors 102 and/or one or more medical devices 104. For convenience, illustrative embodiments may refer to receiving data from one or more sensors and/or one or more medical devices generally as receiving data from the sensors 102. However, it should be understood that discussion of receiving data from the sensors 102 is intended to also include receiving data from the medical devices 104.

To reiterate, the device interface 106 receives/streams real-time patient data from the sensors 102. The device interface 106 may receive data from a variety of sensors 102, such as blood oximeter, a blood pressure measurement device, a pulse measurement device, a glucose measuring device, one or more analyte measuring devices, and/or an electrocardiogram recording device, amongst others. In some embodiments, the device interface 106 simultaneously communicates with a plurality of sensors 102 and/or medical devices. Accordingly, the device interface 106 may aggregate and/or compile the various received patient data.

The system 100 includes a database 105 having access to the patient EHR 103, as described previously. FIG. 4B schematically shows details of the interaction between the EHR 103 and the database 105 in accordance with illustrative embodiments. In various embodiments, the system 100 may communicate with the EHR 103 via a Fast Healthcare Interoperability Resources (FHIR) interface 107.

FHIR is a standard for exchanging healthcare information electronically. FHIR is designed to facilitate the exchange of healthcare data between different healthcare systems, applications, and organizations in a standardized, secure, and interoperable manner. FHIR is closely related to electronic health records (EHRs) in several ways.

For example, FHIR provides a standardized framework for exchanging electronic health information, including data stored within the EHR 103. By using FHIR, the EHR 103 may communicate with other healthcare applications, systems, and devices, enabling seamless data exchange and interoperability. FHIR also promotes interoperability by defining standard data formats and APIs (Application Programming Interfaces) for accessing and exchanging healthcare data. This allows different EHRs 103 to communicate and share patient information more effectively, regardless of the specific technologies or platforms they use.

FHIR also defines a set of resource types and data elements for representing various aspects of healthcare information, such as patients, medications, allergies, encounters, and observations. The EHR 103 can use FHIR resources to organize and represent patient data in a structured format, making it easier to access, retrieve, and exchange specific pieces of information.

Many modern EHRs 103 support FHIR as a means of interoperability and data exchange. They may provide FHIR APIs that allow external applications to access and interact with patient data stored within the EHR. This enables the FHIR interface 107 to integrate seamlessly with the EHRs 103 and leverage the data stored within them. In various embodiments, the FHIR interface 107 may include a graphical button or option to “push protocol to EHR.” Alternatively, the EHR 103 may be updated automatically as new protocols 120 are generated. This allows the protocol 120 to be input or updated within the EHR 103.

In various embodiments, the system 100 may be integrated with the EHR 103 such that the system is accessible via single sign-on 109 (SSO 109). SSO 109 allows users to access multiple healthcare applications and systems with a single set of login credentials. Instead of requiring users to remember and enter separate usernames and passwords for each application or system they need to access, SSO 109 streamlines the authentication process by providing a centralized authentication mechanism. Because medical professionals often need to access various clinical applications, diagnostic systems, and patient management tools, SSO 109 can significantly enhance workflow efficiency and user experience.

Advantageously, illustrative embodiments may distribute and assign alerts using the best practice alerts 111 (BPA) of the electronic health record 103 (e.g., via the FHIR interface 107). The BPA 111 in the EHR 103 notifies healthcare providers of potential issues or opportunities for improvement in patient care based on established best practices, guidelines, or protocols. BPAs 111 are integrated into the EHR 103 systems to assist clinicians in making informed decisions at the point of care, thereby enhancing patient safety, quality of care, and adherence to evidence-based practices.

In various embodiments, examples of BPAs 111 in EHR 103 systems include:
Alerting providers to potential drug-drug interactions or allergies when prescribing medications.
Reminding providers to order recommended preventive screenings or vaccinations based on the patient's age, gender, and medical history.
Notifying providers of abnormal laboratory results or vital signs that require further evaluation or intervention.
Flagging instances of duplicate orders, excessive dosages, or inappropriate medication regimens.
Advising providers on evidence-based clinical guidelines for managing specific conditions or clinical scenarios.
   Advantageously, as described further below, illustrative embodiments enable the discovery and implementation of new medically relevant protocols, and any of the protocols may be integrated into the BPAs 111 of the EHR 103. Thus, the BPA 111 may distribute out notifications/alerts generated by various embodiments of the system 100.

Returning to FIG. 4A, the database 105 may also communicate with the device interface 106 to store the real-time data as it is received via the device interface 106. The database may also include a medical protocol library containing information relating to a plurality of medical protocols. As an example, the medical protocol may include an extubation readiness trial protocol, ventilation vasoactive weaning protocol, sepsis management protocol, enhanced recovery after surgery (ERAS) protocols, and/or other hospital protocols. The information in the library 108 includes eligibility rule and compliance rule for each of the protocols, which are discussed in more detail further below. The medical protocol library may also include specific subscription rule and/or notification rule for each protocol, which are discussed in more detail further below.

A protocol eligibility and concordance module 110 is configured to determine patient eligibility for at least one of the medical protocols as a function of the received patient data. Additionally, the eligibility and concordance module 110 may access historic data (e.g., from the EHR 103 or from the database 105) to determine patient eligibility. To that end, the eligibility and concordance module 110 may communicate with, among other things, the device interface 106, the database 105, and the library 108. The eligibility and concordance module 110 may also sometimes be referred to as the eligibility module 110 and/or the concordance module 110.

Generally, patient eligibility depends on one or more measurements of patient data meeting a threshold status (e.g., above or below a given threshold, or within a particular range) according to the requirements of the protocol stored in the library (e.g., a certain FiO2 and/or BPM measurement). When the patient 101 is eligible for a protocol, the eligibility module 110 triggers an indication that the patient 101 is eligible. In some embodiments, the eligibility module 110 sounds an alert and/or communicates with a user interface 112 to visually indicate the patient is eligible. For example, the eligibility module 110 may prompt a notification on the medical practitioner's device (e.g., smartphone) indicating that the patient is eligible for the protocol. In some embodiments, the user interface 112 also communicates the amount of time the patient has been eligible. Thus, illustrative embodiments advantageously provide real-time updates as to patient eligibility status.

The real-time data collection and eligibility updates contrast to prior art methods that rely on medical practitioners checking, collecting, aggregating, and analyzing a plurality of static patient parameters to determine protocol eligibility. Eligibility rule and/or compliance rule relating to a particular protocol may vary by treatment center (e.g., extubating readiness trial may have different eligibility criteria from hospital to hospital). Furthermore, medical practitioners are limited by their time working a particular shift, and their attention is largely divided examining a number of patients 101. By providing real-time collection and aggregation of multiple data streams from patient-coupled devices 102, illustrative embodiments advantageously provide potentially life-saving clinical response speeds. In various embodiments, one or more medical practitioners determined by the system 100 as being on-call and subscribed to a particular patient may be notified of the real-time eligibility (or compliance state). Accordingly, swift action may be taken to improve patient 101 outcomes. Additionally, or alternatively, various embodiments may automatically enroll the patient 101 in the protocol.

The determination of eligibility is based on a collection of measurement thresholds, events, and/or other binary variables. For example, the eligibility rule for a vasoactive weaning protocol may include that the patients are under cardiac service (determined by the hospital ADT stream or EHR), are on medication drip (another event), have IDO2 index less than a threshold value, and have blood pressure greater than another threshold value.

The system 100 may include a quality reporting module 114 that tracks the time elapsed from patient eligibility until a user acknowledges patient eligibility (e.g., medical practitioner interacts with notification). Alternatively, or additionally, the quality reporting module 114 may track the time elapsed from patient eligibility until a medical practitioner begins the course of action associated with the eligible protocol for the patient 101. By tracking this information, medical practitioner performance may be objectively measured (e.g., which medical practitioners are the fastest responders and what are the impacts of response time on patient 101 clinical outcomes for given protocols).

The system 100 may also include a protocol trigger module 116 that instructs the eligibility and concordance module 110 to begin tracking patient 101 compliance data. The protocol trigger module 116 is configured to receive information regarding the initiation of a course of action in the protocol 120. In various embodiments, the medical practitioner may initiate the course of action (e.g., based on the feedback from the eligibility module 110 indicating the patient 101 is eligible) and inform the protocol trigger module 116 (e.g., through the user interface 112) of the course of action initiation. In some embodiments, the protocol trigger module 116 is configured to automatically initiate the course of action by communicating with the treatment device 104 (e.g., after a medical practitioner approves the initiation via the user interface). In other embodiments, the protocol trigger module 116 can automatically detect that the practitioner has initiated the course of action and initiate tracking concordance data. For example, in the ERT protocol, when the system 100 detects that the ventilator mode has been changed, this may be used to detect the start of an ERT protocol and trigger dynamic concordance tracking for the protocol.

After the course of action for the protocol is initiated, the concordance module 110 may continue to communicate with the same, different, or additional sensors 102 to determine patient 101 compliance with the protocol. The concordance module 110 may determine the dynamic concordance of the patient 101 with the medical protocol as a function of newly received patient data and the compliance rule in the protocol library. Compliance with a particular eligibility rule or compliance rule may be determined by calculating, comparing, measuring, and/or otherwise determining whether a patient parameter meets the criteria in the protocol library for the particular protocol (e.g., whether a condition is satisfied). To that end, in various embodiments, the patient variable is compared to the level or range of the compliance rule for the protocol in the library 108. In various embodiments, the dynamic concordance rate is the rate at which patient data (e.g., measured, hidden internal state variable calculation, and/or otherwise determined) is consistent with the compliance rule 124 of the protocol over time. Thus, an overall dynamic concordance rate may be determined for the patient on the protocol. In some embodiments, parameter specific dynamic concordance rates may be calculated for each parameter of the compliance rule 124 (e.g., the two parameters in the compliance rule 124 of FIG. 2).

Additionally, the concordance module 110 may calculate and visually indicate (e.g., via the user interface 112) the real-time patient dynamic concordance status. FIG. 4A schematically shows eligibility rule 122 and compliance rule 124 for an example of an extubation readiness test protocol in accordance with illustrative embodiments. It should be understood that the tracked parameters for eligibility may differ from the tracked parameters for compliance. In the example of FIG. 2, the eligibility rule 122 have three separate variables/parameters, and the compliance rule 124 have two separate variables/parameters. The concordance module 110 may calculate a dynamic concordance rate for the overall compliance rule 124. Additionally, or alternatively, the concordance module 110 may calculate a dynamic concordance rate for each individual parameter. The concordance module 110 sends the dynamic concordance rate to the user interface 112, which displays the dynamic concordance rate. Accordingly, the attention of the medical practitioner may be drawn to the patient 101 in need of action (e.g., having a low or trending downward dynamic concordance rate).

The quality reporting module 114 may track a dynamic concordance rate as a percentage of time over which the patient 101 is compliant with the medical protocol. The quality reporting module 114 may also track the patient eligibility time. Additionally, the quality reporting module 114 may retrospectively look at patient concordance rate and/or eligibility time to see how well a particular patient and/or patients of a particular medical practitioner have performed relative to a patient population. Additionally, the reporting module 114 may be configured to display the received patient data and to mark non-compliant portions of the received data. Thus, the quality reporting module 114 allows medical facilities and/or staff to evaluate how well patients were managed retrospectively. For example, if eligibility times are very high, medical practitioners become aware of it. This assists with hospital policy formation and also with ensuring prompt action by the medical staff.

The system may include a patient tracking module 118 configured to receive the subscription rule 128 and the notification rule 130, as well as patient status information from the protocol eligibility and concordance module 110. As the rule 130 are met, the tracking module 118 sends a notification to the various subscribed users in accordance with their subscription level. For example, notifications may be sent when eligibility status is changed, when the course of action begins (also referred to as protocol enrollment), and/or when the patient is out of compliance.

The system of FIG. 4A may include a risk-based monitoring engine 1000 (also referred to as “risk engine 1000”) configured to receive data from bedside monitors 102, electronic health records 103, treatment devices 104, and any other information that may be deemed relevant to make an informed assessment regarding the patient's clinical risks, and any combination thereof of the preceding elements.

In illustrative embodiments, the risk engine 1000 may include a physiology observer module 119 that utilizes multiple measurements to estimate probability density functions (PDF) of internal state variables (ISVs) that describe the components of the physiology relevant to the patient treatment and condition in accordance with a predefined physiology model. The JSVs may be directly observable with noise (as a non-limiting example, heart rate is a directly observable ISV), hidden (as a non-limiting example, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV).

In some embodiments, when the physiology observer module 119 evaluates a set of ISVs at a given time step (e.g., tk; tk+1; generally tk+n), the system 100 may not have a complete set of ISV measurements contemporaneous with that given time step. For example, the system 100 may have measurements for that given time step for some internal state variables, but may not have measurements for that given time step for some other internal state variables (e.g., a contemporaneous measurement for an intermittent ISV may not be available for the given time step). Consequently, that intermittent ISV is, for purposes of evaluating ISVs at the given time step, a hidden ISV. However, evaluation of the set of ISVs by the physiology observer module 119 (as described herein) is nevertheless possible according to embodiments described herein because the predicted PDFs of ISVs 211 carry in them the influence of past measurements of that intermittent ISV, and consequently those predicted PDFs of ISVs 211 are, in illustrative embodiments, sufficient input for the physiology observer module 119.

In one embodiment, instead of assuming that all variables can be estimated deterministically without error, the physiology observer module 119 of the present disclosure provides probability density functions as an output. Additional details related to the physiology observer module 119 are provided herein.

The clinical trajectory interpreter module 123 may be configured, for example, with multiple possible patient states, and may determine which of those patient states are probable and with what probability, given the estimated probability density functions of the internal state variables. Examples of particular patient states include, but are not limited to, hypotension with sinus tachycardia, hypoxia with myocardial depression, compensated circulatory shock, cardiac arrest, hemorrhage, amongst others. In addition, these patient states may be specific to a particular medical condition, and the bounds of each of the patient states may be defined by threshold values of various physiological variables and data. In various embodiments, the clinical trajectory interpreter module 123 may determine the patient conditions under which a patient may be categorized using any of information gathered from reference materials, information provided by health care providers, other sources of information.

The reference materials may be stored in the database 105 or other storage device that is accessible to the risk-based monitoring application 1020 via network interface 113, for example. These reference materials may include material synthesized from reference books, medical literature, surveys of experts, physician provided information, and any other material that may be used as a reference for providing medical care to patients. In some embodiments, the clinical trajectory interpreter module 123 may first identify a patient population that is similar to the subject patient being monitored. By doing so, the clinical trajectory interpreter module 123 may be able to use relevant historical data based on the identified patient population to help determine the possible patient states.

The clinical trajectory interpreter module 123 is capable of also determining the probable patient states under which the patient can be currently categorized, given the estimated probability density functions of the internal state variables, as provided by physiology observer module 119. In this way, each of the possible patient states is assigned a probability value from 0 to 1. The combination of patient states and their probabilities is defined as the clinical risk to the patient.

Additional details regarding the risk-based patient monitoring engine 1000 and calculation of hidden internal state variables are described in U.S. patent application Ser. Nos. 17/033,591, and 17/501,978, each of which is incorporated by reference herein in its entirety.

It should be appreciated in view of the foregoing that proper establishment of appropriate medical protocols 120 is significant to overall patient 101 management and welfare. For many medical procedures, unified standards for protocols 120 are not established. For example, the eligibility rule 122, compliance rule 124, protocol trigger rule 126 (also referred to as the protocol trigger 126), subscription rule 128, and/or notification rule 130 of any given protocol 120 (e.g., an ERT protocol 120) may vary from hospital to hospital. Generally, the protocol 120 is built around expertise and preference of medical staff at the given clinical environment.

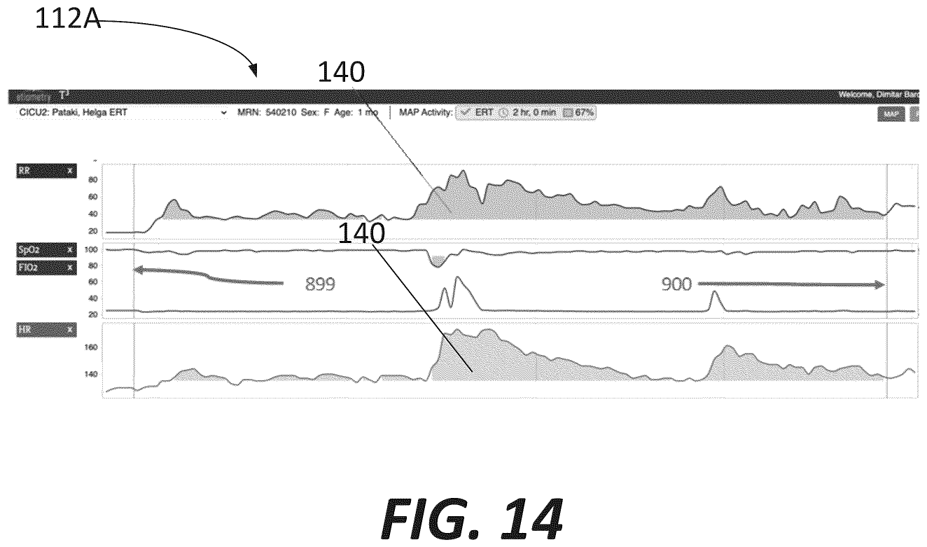

In practice, both generating the protocol 120 and integrating the protocol 120 with the system 100 (e.g., to receive streamed data and to track eligibility/compliance) are difficult and time consuming. Illustrative embodiments advantageously provide a configuration user interface 112 that allows the medical practitioner to visually generate and/or edit the medical protocol 120 within the graphical user interface, with little or no coding. This innovation allows point of care adjustments to the protocol 120 in real-time and also allows the medical practitioner to optimize the protocol 120 by simulating with historic data acquired by the system 100 (e.g., from the same clinical environment or from a different clinical environment). Furthermore, in various embodiments, the interface for generating the protocols may be part of, or displayed on the same monitor, as the active interface 112A that displays patient data (e.g., as shown in FIG. 3 and FIG. 14). Alternatively, the interface for generating protocols may be different from the interface 112.

For the sake of clarity, the active interface 112A used when the protocol is deployed in a medical setting (e.g., as shown in FIGS. 3, 11, and 14) may be referred to as the active interface 112A. The interface 112 used for generation of the protocol 120 may be referred to as the configuration interface 112. The active interface 112A and the configuration interface 112 may be, but are not necessarily, part of the same interface. Furthermore, the active interface 112A and the configuration interface 112 may be, but are not necessarily, viewed on the same device. For simplicity purposes, the two interfaces are described herein as being part of the same interface 112 (e.g., on the same display device). However, in various embodiments these interfaces 112A and 112 may be separate (e.g., on different displays).

FIG. 4C schematically shows the system 100 for generating and simulating medical protocols 120 in accordance with illustrative embodiments. Among other things, the system 100 includes a database 105 that stores real-time patient data received from the sensors 102 and/or medical devices 104. The database may also have access to the patient EHR 103. Additionally, the database 105 may contain test and/or sample data (e.g., from a blood test) related to one or more patients 101.

The database 105 may be the same database 105 shown in FIG. 4A. The database 105 may store patient data as it is collected by, or input into, the system 100 (e.g., during system 100 operation, as described in U.S. patent application Ser. No. 17/502,005). The patient data may thus be from a specific clinical use environment (e.g., a particular hospital or unit of a hospital). However, in some embodiments, the patient data may be pooled from multiple clinical use environments (e.g., a collection of data from multiple hospitals collected from various systems 100). Accordingly, when a protocol 120 is simulated using historic data, this historic data may be drawn from the database 105, among other sources. However, some embodiments may also include non-patient generated data (e.g., simulated data, hypothetical data, AI generated data, etc.).

The database 105 may also include a medical protocol library containing information relating to one or more medical protocols. As an example, the medical protocol may include information relating to an extubation readiness trial protocol, ventilation vasoactive weaning protocol, sepsis management protocol, enhanced recovery after surgery (ERAS) protocols, and/or other hospital protocols. The information in the database 105 includes eligibility rule 122 and compliance rule 124 for each of the protocols 120. The database 105 may also include protocol trigger rule 126, subscription rule 128 and/or notification rule 130 for each protocol.

In various embodiments, the system 100 may include a data de-identifier 140 that communicates with the database 105. Because the system 100 may run a simulation using real historic patient data, it may be advantageous to de-identify the patient data (i.e., disassociate the patient identification from the data). The communication between the data de-identifier 140 and the database 105 may be a two-way communication, such that the patient data in the database 105 becomes de-identified.

The system 100 includes a protocol simulator 142 that communicates with the database 105 and/or the graphical compiler 146. The protocol simulator 142 receives details of one or more protocols 120 (e.g., the various rules and conditions of the protocol) from a stored protocol in the database 105, or from a generated protocol from the graphic compiler 146. The simulator 142 also takes patient data from the database 105 and simulates the outcomes of the received protocol(s) 120 using the patient data.

For example, the simulator 142 may be part of the system 100 in the medical surgical ICU in Boston Children's Hospital. The database 105 may have access to patient data from all patients in the unit within the last 12-months. The patient data may be de-identified by the de-identifier 140. The simulator 142 may then simulate how the received protocol 120 would have functioned in a particular unit based on the patient data from that unit.

As an example, the simulator 142 may simulate how often patients 101 are eligible for the protocol 120, how often patients 101 are compliant with the protocol 120, the dynamic concordance rate for the patients 101, and compare the outcomes for patients 101 who have high compliance on a given protocol versus low compliance on the given protocol. This information may be used to iterate the protocol 120 using the graphical compiler 146 as described further below.

Various embodiments provide a graphical compiler 146 that allows for graphical generation and/or modification of the protocol 120. As discussed previously, the graphical compiler 146 allows a user to create a new clinical protocol using a graphical interface. Instead of writing code that describes the protocol, or in addition to writing code, the user may select graphical objects pre-configured with various protocol 120 requirements.

The user may place the graphical objects in a certain sequence, and may also edit values for parameters associated with the graphical objects in an adjacent parameter window, thereby creating a new clinical protocol 120. Instead of writing a code, the user may advantageously graphically describe the protocol 120, and has a window for providing specific parameter values (e.g., using drop-down menus). Accordingly, the graphical compiler 146 allows the user to compile a MAP in a graphical way.

The simulator 142 communicates with a protocol analyzer 144. The analyzer 144 provides reporting (e.g., how often the protocol notifies, what is it expected impact on patient outcomes, etc.). This information can be used as feedback to iterate the protocol 120 (e.g., using the graphic compiler 146 to change some of the settings of the protocol). Users may keep iterating until they are satisfied with the patient outcomes associated with the particular protocol 120 (e.g., with a given level of compliance with the protocol). The protocol 120 may then be deployed to the production platform 148 at the clinical site.

It should be apparent that the various components of the system 100 and sub-system 100B my communicate with one another. The arrows in FIG. 4C are shown as an example of a communication workflow in accordance with illustrative embodiments. However, some embodiments may not include some components (e.g., a data de-identifier 140), and/or may combine some components (e.g., the simulator 142 and analyzer 144). As another example, the analyzer 144 may be combined with the quality reporting module 114.

The system 100 advantageously allows practitioners to deploy medical protocols quickly with an expectation of success for clinical outcomes. For example, the medical practitioner will have an understanding of how actionable certain protocols are, how often they need interaction, and what need to be change in the practice to improve clinical outcomes.

FIGS. 4A-4C schematically show details of the system 100 of FIG. 1 configured in accordance with illustrative embodiments of the invention. Each of these components is operatively connected by any conventional interconnect mechanism. FIGS. 4A-4C simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIGS. 4A-4C only schematically show each of these components. Those skilled in the art should understand that each of these components can be implemented in a variety of conventional manners, such as by using hardware, software, or a combination of hardware and software, across one or more other functional components. For example, the simulator 142 (discussed in detail below) may be implemented using a plurality of microprocessors executing firmware. As another example, the compiler 146 may be implemented using one or more application specific integrated circuits (i.e., “ASICs”) and related software, or a combination of ASICs, discrete electronic components (e.g., integrated circuits), and microprocessors. Accordingly, the representation of the simulator 144 and other components in a single box of FIGS. 4A-4C is for simplicity purposes only. In fact, in some embodiments, the simulator 144 of FIGS. 4A-4C is distributed across a plurality of different components—not necessarily within the same housing or chassis.

It should be reiterated that the representation of FIGS. 4A-4C is a significantly simplified representation of the system 100. Those skilled in the art should understand that such a device has other physical and/or functional components, such as central processing units, other packet processing modules, and short-term memory. Accordingly, this discussion is not intended to suggest that FIGS. 4A-4C represent all of the elements of the system 100. Indeed, much of what was described with regard to FIGS. 4A-4C can also be applied to components of the system 100 of FIG. 1.

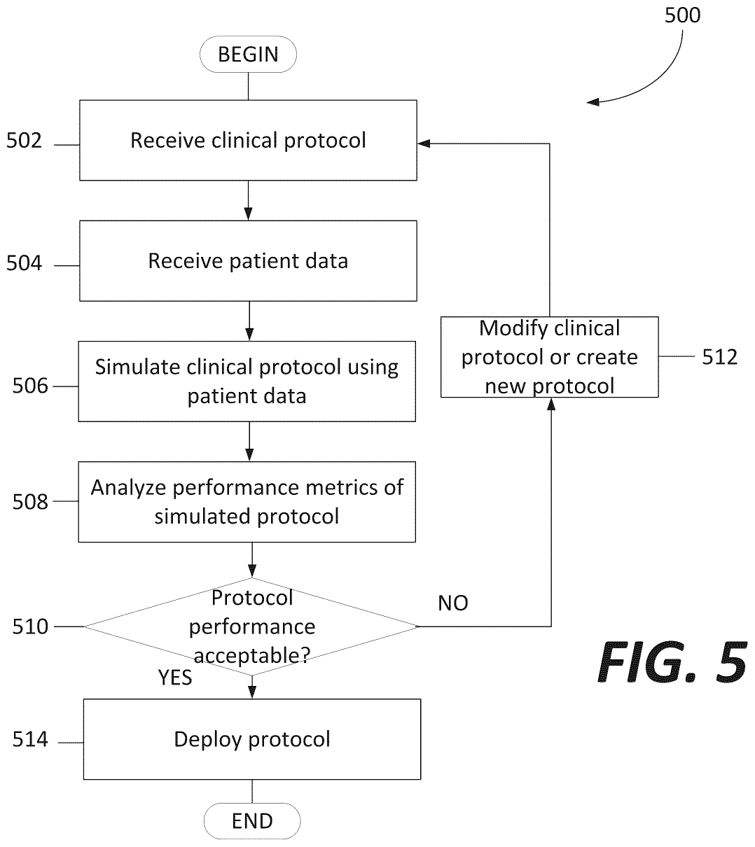

FIG. 5 shows a process 500 of simulating the clinical protocol 120 in accordance with illustrative embodiments of the invention. It should be noted that this method is substantially simplified from a longer process that may normally be used. Accordingly, the method shown in FIG. 5 may have many other steps that those skilled in the art likely would use. In addition, some of the steps may be performed in a different order than that shown, or at the same time (e.g., steps 502 and 504). Furthermore, some of these steps may be optional in some embodiments. Accordingly, the process 500 is merely exemplary of one process in accordance with illustrative embodiments of the invention. Those skilled in the art therefore can modify the process as appropriate.

The process 500 begins at step 502, which receives the clinical protocol 120. As mentioned previously, the clinical protocol 120 may be a protocol stored in the database 105 and/or received from the graphic compiler 146. In some embodiments, more than one clinical protocol 120 may be received by the simulator 142 at a time. The received clinical protocol 120 may be similar to the protocol 120 shown in FIG. 2. Among other things, the clinical protocol 120 may include an eligibility rule 122, compliance rule 124, protocol trigger 126, subscription rule 128, and/or notification rule 130. Each rule and/or trigger may have one or more conditions. The protocol 120 may include one or more eligibility criteria 115, compliance criteria 115, trigger criteria 115, subscription criteria 115, and subscription criteria 115.

The process then proceeds to step 504, where the simulator 142 receives patient data. The received patient data may be stored in the database 105. In various embodiments, the patient data is real historic patient data from the same clinical setting (e.g., the same unit in the same hospital). The user may select the timeframe from which the data is drawn. For example, data may be used from the prior 6-months, prior 12-months, or between a particular window (e.g., from date X to date Y). For example, the patient data may be selected over a particular time window. One skilled in the art may filter the patient data on demographic basis (e.g., age, sex, race, weight), health status (e.g., patient has heart disease, patient has COVID, patient recently had surgery), and/or event basis (e.g., from the onset of COVID to the release of the vaccine, etc.). One skilled in the art may filter the patient data to simulate the operation of the clinical protocol 120 on a desired patient population. Thus, the received patient data may relate to a specific parameter collected over a period of time from one or more sensors and/or medical devices. In some embodiments the data may include live data collected in real time, as well as data from the database 105, and/or the EHR 103.

At step 506, the process 500 simulates the clinical protocol 120 using the received patient data. The simulator 142 may compare any of the details of the protocol 120 with the patient data. As an example, the simulator 142 may compare the compliance rule 124 of the received protocol 120 with the patient data over the last 12-months. Additionally, or alternatively, the simulator 142 may compare the eligibility rule 122 of the protocol 120 with the patient data for specific types of patients (e.g., patients having heart disease). The simulator 142 may simulate all aspects of the protocol 120, including simulating the notification conditions, as well as the set reporting conditions. As will be discussed further below, various reporting conditions, such as conditions represented by graphical objects 150 in the adoption, adherence, impact, and/or potential sub-menu may be simulated. The simulator 142 may pass the results to an analyzer 144 to analyze performance metrics of the simulated protocol 120.

At step 508, the performance metrics of the simulated protocol are analyzed. To that end, the simulator 142 communicates with the analyzer 144. The analyzer 144 is configured to provide desired performance metrics of the simulated protocol 120 on the patient data. The analyzer 144 takes the results of the simulation and provides them to the medical practitioner/administrative staff in an easily digestible manner. For example, the simulator 142 may determine that a first population of the patients from the patient data are 100% compliant with the protocol, and that a second population of patients from the patient data are 50% compliant with the protocol. Preferably, the first population and the second population are demographically similar (e.g., age, medical condition, etc.) or have been selected beforehand to have some common medical history (e.g., patients of the same oncology ward). The analyzer 144 may indicate that the first population of patients remained in the ICU for 2 days, and that the second population remained in the ICU for 5 days. Thus, the medical practitioner can determine that high-compliance patients 101 have a reduced ICU stay relative to low-compliance patients 101.

To that end, the analyzer 144 may include an event detector 149 configured to detect various patient events by automatically analyzing the data received from the sensor/medical device interface 106 and/or from the protocol simulator 142. The event detector 149 may include a function library having one or more functions configured to analyze the data and determine the event (as described further below).

Accordingly, the medical practitioner may choose to adjust hospital procedures or to direct hospital resources to drive patient compliance towards 100%, thereby providing targeted outcomes based on historic patient performance. In the above example, the target outcome is reducing average length of ICU stay by 3 days when converting low compliance patients to high compliance patients. At the very least, illustrative embodiments highlight the potential for improvement based on compliance with the protocol.

The simulator 142 may also show how often active notifications are sent in accordance with the various conditions 115 of the subscription rule 128 (e.g., to the respiratory therapist or to the nurse). In various use cases, it may be beneficial to have a protocol 120 that sends 2-5 notifications per day, not thousands. In various embodiments, the simulator 142 simulates all aspects of how the protocol 120 functions at any given time for each of the patients in database 105. Thus, the medical practitioner may use the graphical compiler 146 to adjust or create a new protocol 120.

The process then proceeds to step 510, which asks if the protocol 120 performance is acceptable. The protocol 120 performance may be acceptable, for example, if high compliance leads to beneficial clinical outcomes. As yet another example, the protocol 120 performance may be unacceptable if the number of notifications sent to medical staff are under some threshold number. Conversely, the protocol 120 performance may not be acceptable if high compliance does not lead to beneficial clinical outcomes, and/or if the number of notifications are too high. What qualifies as acceptable protocol 120 performance may vary from medical practice to medical practice.

If the protocol 120 performance is unacceptable, the process proceeds to step 512, which modifies the clinical protocol 120. For example, the simulator 142 may simulate a second given protocol 120. The analyzer 144 may determine that a first population of the patients were 100% compliant with the protocol 120, and that a second population of patients were 50% compliant with the protocol 120. The analyzer 144 may indicate that the first population of patients stayed in the ICU for 3 days, and that the second population stayed in the ICU for 3 days. Thus, the medical practitioner can determine that compliance variance for the protocol 120 does not have a beneficial impact on clinical outcomes (e.g., at least between 50% and 100%). Accordingly, the medical practitioner may deem the protocol performance to be unacceptable, and may choose to proceed to step 512, which adjust the second given protocol 120 and/or creates a new protocol 120 (e.g., by using the graphic compiler 146). Steps 502-510 are then repeated. It should be apparent that in step 502, the received clinical protocol is the new protocol. In step 504, the patient data may be the same patient data received previously. Therefore, in some embodiments, the process may be shown as going directly to step 506.

If at step 510 the protocol is acceptable, the process proceeds to step 514, which deploys the protocol. When the protocol 120 is deployed, the protocol 120 is applied to real patients in real-time (e.g., on-site at the medical facility using the system 100). The various sensors 102 and medical devices 104 track relevant patient data and provide feedback on eligibility. Accordingly, the system 100 may begin tracking patient eligibility and/or compliance for the deployed clinical protocol 120. The process then comes to an end.

It should be apparent that the above described process provides a number of advantages. As mentioned previously, the protocol 120 may be, for example, hospital specific or unit specific. Therefore, illustrative embodiments advantageously enable customization of protocols 120 based on unit (e.g., cardiac ICU, general ICU) and/or specific patient demographics. Illustrative embodiments advantageously allow medical practitioners to easily create, test, modify, and deploy new clinical protocols.

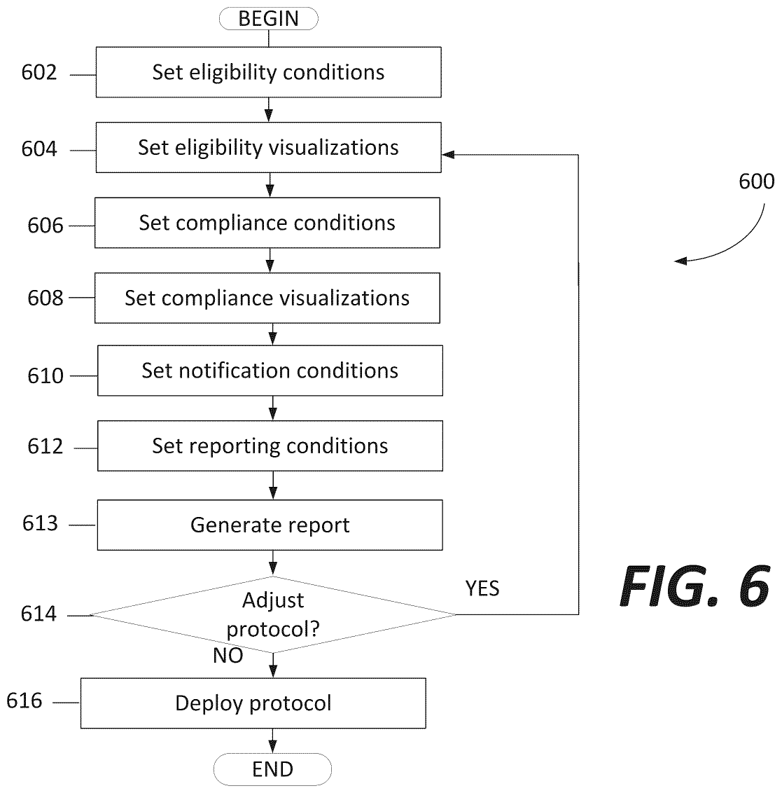

FIG. 6 shows a process of generating the clinical protocol using the graphic compiler 142 in accordance with illustrative embodiments of the invention. It should be noted that this method is substantially simplified from a longer process that may normally be used. Accordingly, the method shown in FIG. 6 may have many other steps that those skilled in the art likely would use. In addition, some of the steps may be performed in a different order than that shown, or at the same time. Furthermore, some of these steps may be optional in some embodiments. Accordingly, the process 600 is merely exemplary of one process in accordance with illustrative embodiments of the invention. Those skilled in the art therefore can modify the process as appropriate.

The process 600 begins at step 602, which configures the eligibility rule 122. The eligibility conditions 155 may come in a variety of types, and may also include specific parameters. To that end, the graphical compiler 146 may provide the configuration user interface 112 pre-populated with a variety of selectable graphical condition objects 150. The user interface 112 may be provided on, among other things, a touch-screen display (e.g., on a hospital monitor, mobile device, smartphone, etc.).

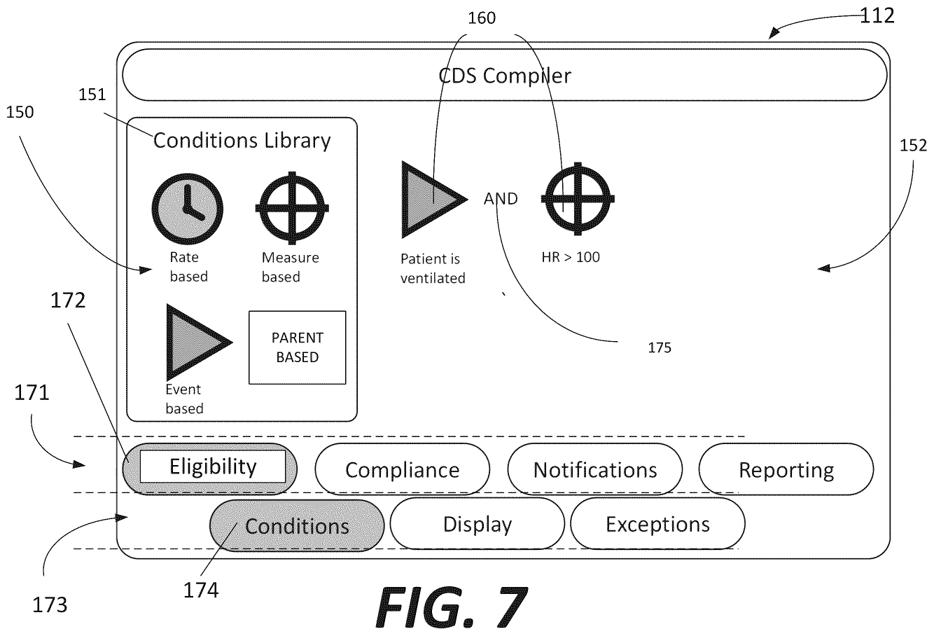

FIG. 7 schematically shows a user interface 112 of the graphical compiler 142 in accordance with illustrative embodiments of the invention. The user interface 112 has options for setting all of the various conditions associated with the protocol 120. In the specific view of FIG. 7, the interface 112 shown is for selecting eligibility conditions 115.

To arrive at the eligibility menu, the user may select the eligibility button 172 in the main menu 171. The sub-menu 173 displays items associated with the selected menu item (e.g., conditions, display, and exceptions items). The “conditions” item 174 is selected in the sub-menu 173 shown in FIG. 7.

In this view, to configure the eligibility conditions 115, the user may select one or more graphical objects 150 to define one or more conditions 115 from the conditions library 151. Among other things, the graphical object 150 may be a rate based object 150, a measurement based object 150, an event based object 150, and/or a parent based object 150. Various combinations of objects 150 may be used to select eligibility conditions 115 for the protocol 120. For example, as shown in FIG. 7, the eligibility conditions 115 for the protocol 120 may include that the patient 101 is ventilated (an event based object) and the patient's 101 heart rate is above 100 (measurement based object). Thus, the user may select the event based object 150 and the measurement based object 150 to populate those objects 150 in a simultaneously displayed sequence window 152. The sequence window 152 shows all the selected graphical objects 160 of the eligibility rule 122. The selected graphical objects 150 becomes the eligibility conditions 115 of the eligibility rule 122. Accordingly, after the protocol 120 is deployed, if the patient 101 meets the eligibility criteria set in the sequence window 152, the patient 101 becomes eligible for the protocol 120.

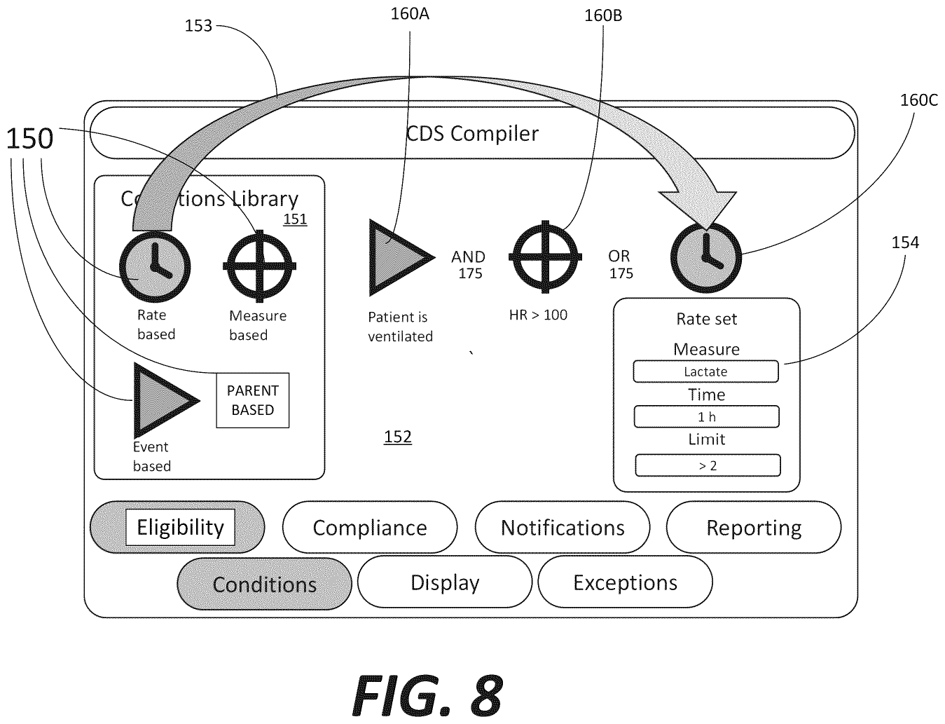

As shown, the selected graphical objects 150 may include a logical operator 175 that allows further customization of the rule. For example, in various embodiments the logical operator may include “and”, “or”, “and/or”, “not”, etc. In FIG. 7, the logical operator is an “and” operator. Additionally, each graphical object 150 may include its own additional input values to help define the condition. In various embodiments, the additional input values (e.g., patient is ventilated, HR>100) may be input directly into the sequence window 152 (e.g., in a parameter window 154 shown in FIG. 8).

FIG. 8 schematically shows the user interface of FIG. 7 changing a condition of the protocol 120 using the graphical compiler 146. As shown, the user may select the condition object 150 from the conditions library 151 to move 153 the object 150 into the sequence window 152 (e.g., drag and drop the object 150). This allows the user create or adjust the protocol 120. For example, FIG. 8 shows the user adding a rate based condition 150 using the compiler 146. In the example of FIG. 8, the eligibility rule for the protocol now includes the 1^{st} condition 115 that the patient is ventilated (selected event based object 160A), and the 2^{nd} condition 115 that the patient's heart rate is above 100 (selected measurement based object 160B) or the 3rd condition 115 patient's lactase increase 2 mmol/L per hour (selected rate based object 160C).

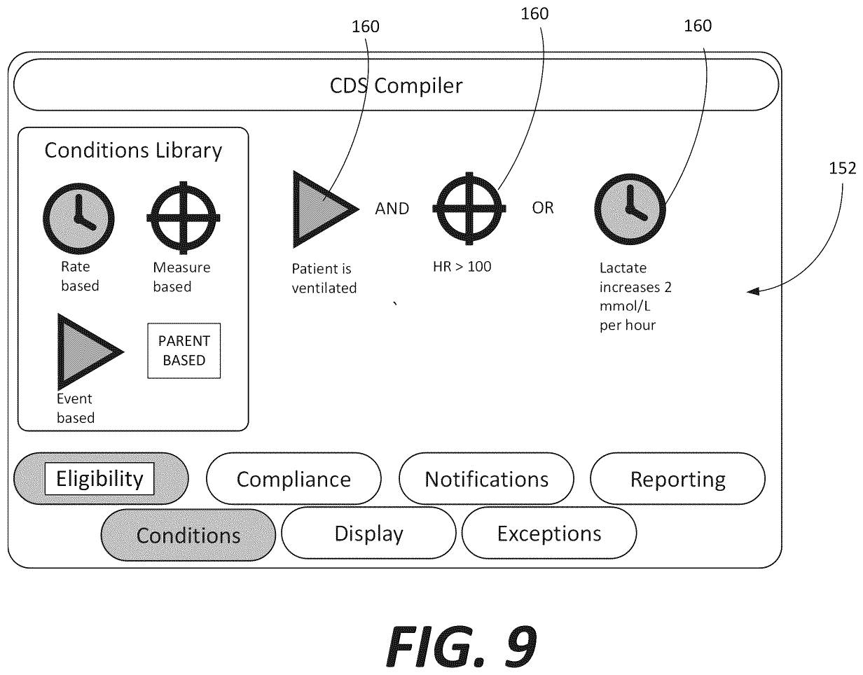

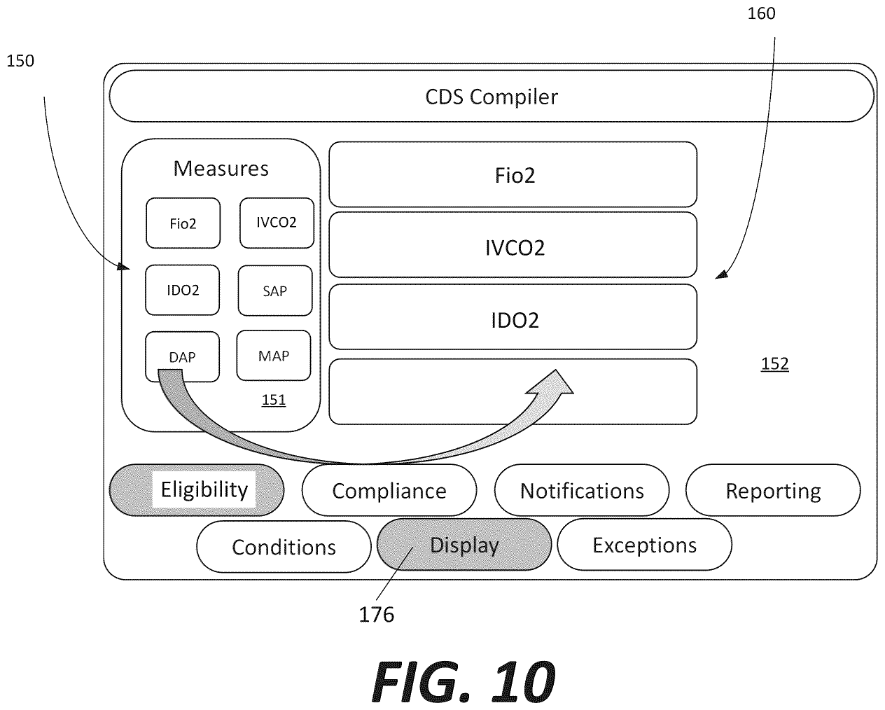

After selecting the graphical object 150 from the library 151, the graphical object is moved into the protocol sequence window 152 and becomes a selected graphical object 160. In various embodiments, the selected graphical object 160 may be configured to receive specific parameters or requirements based on the type of selected graphical object 160. To that end, a parameter window 154 may appear as an overlay or adjacent to the corresponding graphical object 160. The parameter window 154 receives inputs from the user relating to the condition 115. Based on the type of selected object 160, the window 154 may come pre-populated with specific parameter requests. For example, the parameter window 154 for the rate based graphical object 160C may have inputs for what to measure, how long to measure for, and a limit over that time period. In the example of FIG. 8, the user inputs that the lactate rate is greater than 2 mmol/L per 1 hour into the parameter window 154. Accordingly, as shown in FIG. 9, the eligibility conditions 115 are set for the protocol 120 using the user interface of the graphical compiler 146. The process then proceeds to step 604, which sets the visualizations associated with eligibility. The visualizations are what the active interface 112 shows to the medical practitioner when the patient 101 is eligible. FIG. 10 schematically shows the configuration interface 112 for selecting display visualizations for the active interface 112 in accordance with illustrative embodiments. The visualizations for eligibility may be different from the conditions. However, in various embodiments, the eligibility display objects 150 may be selected to match the eligibility conditions 115.

In various embodiments, the user selects the “display” sub-menu item 176 to access the display interface 112. The display interface 112 shows the plurality of selectable graphical display objects 150. FIG. 10 shows six options, but it should be understood that various embodiments may provide more or fewer options. Preferably, the pre-populated library 151 includes a list of display objects 150 corresponding to the selected eligibility conditions 115. The user may select one or more of the various objects 150. As shown in the example of FIG. 10, the user has selected Fio2, IVCO2, and IDO2 display objects 160. The selected display objects 160 are populated into a selected display objects window 152, where the user may remove the objects (e.g., by hitting an “X” icon in a corner, or by swiping the selected display objects off of the screen).

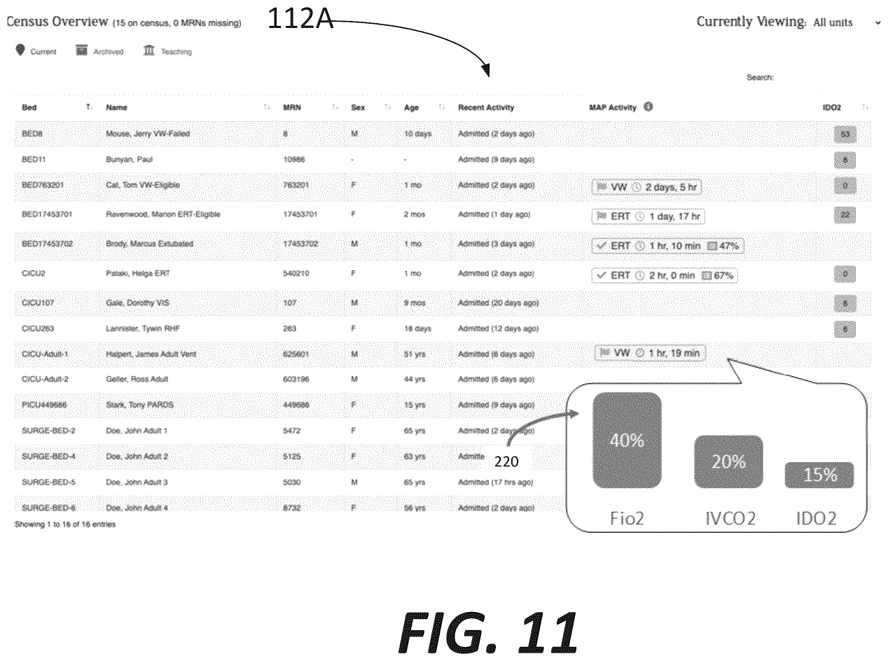

FIG. 11 schematically shows an example of the user interface 112 displaying the selected display parameters 160 of FIG. 10. As shown, from the census overview, selecting a given eligible protocol displays 220 the selected display variables 160 from the objects window 152. Additionally, or alternatively, a more detailed view may be provided with a detailed view of the collected parameters over time (as shown in FIG. 14).

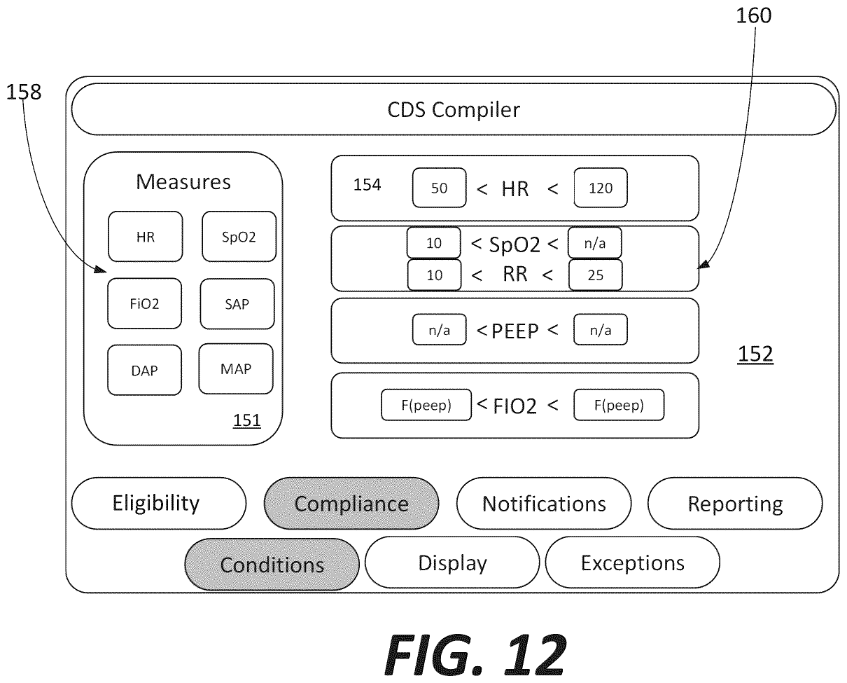

Returning to the process of FIG. 6, at step 606 compliance conditions for the protocol 120 are set. FIG. 12 schematically shows generation of the compliance rule 124 using the user interface 112 of the graphical compiler 146 in accordance with illustrative embodiments of the invention. The interface of FIG. 12 operates similarly to the interface shown in FIGS. 7-9. Thus, step 606 operates similarly to step 602, except that the selected conditions 115 correspond to compliance conditions 115 for the protocol 120 as opposed to eligibility conditions 115. The user selects the desired compliance condition objects from a plurality of graphical compliance objects 158. As the objects 158 are selected, they are populated into the selected compliance conditions window 152. Various conditions include parameter settings that may be input by the user, such as a lower limit and upper limits (e.g., 50 bpm and 120 bpm). The parameter window 154 may be integrated with the selected graphical compliance object 160 (or may be adjacent thereto, as shown in FIG. 8).

After the compliance conditions 115 are set, the process proceeds to step 608, which sets the compliance visualizations for the active interface 112 after the protocol 120 begins, also referred to as the user compliance visualizations. Similar to step 604, the user selects one or more parameters to be displayed while the protocol is ongoing. The user may select the same type of parameters as the compliance conditions, and/or different parameters.

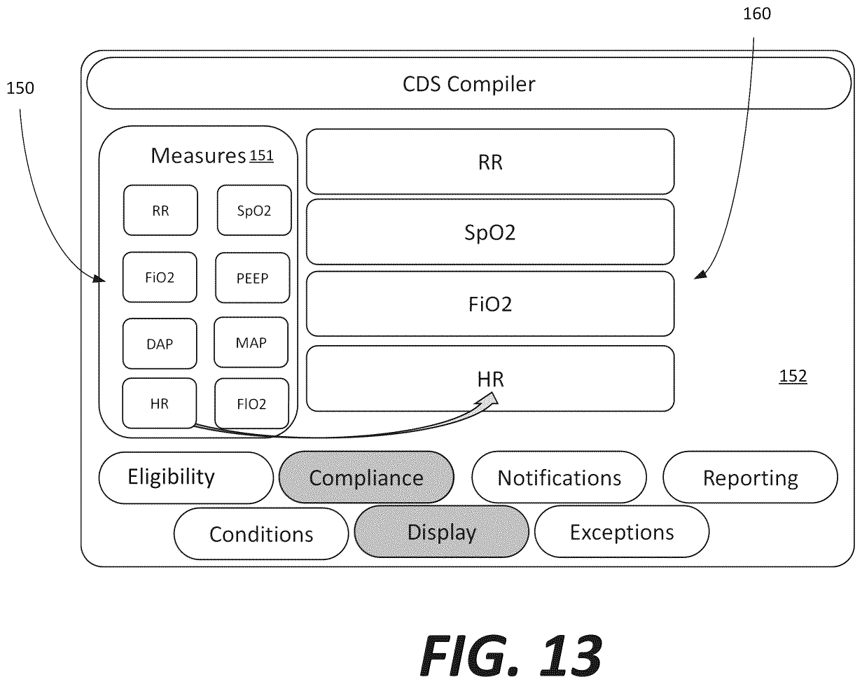

FIG. 13 schematically shows the configuration interface 112 for selecting display visualizations for the active interface 112 in accordance with illustrative embodiments. The visualizations for compliance may be different from the conditions. However, in various embodiments, the compliance display objects 150 may be selected to match the compliance conditions 115.

In various embodiments, the user selects the “display” sub-menu item to access the display interface 112. The display interface 112 shows the plurality of selectable graphical display objects 150. FIG. 13 shows eight options, but it should be understood that various embodiments may provide more or fewer options. Preferably, the pre-populated library 151 includes a list of display objects 150 corresponding to the selected compliance conditions 115. The user may select one or more of the various objects 150. As shown in the example of FIG. 13, the user has selected RR, SpO2, FiO2, and HR display objects 160. The selected display objects 160 are populated into a selected display objects window 152. The user may remove the objects (e.g., by hitting an “X” icon in a corner, or by swiping the selected display objects off of the screen) if they are no longer desired as part of the visualization.

FIG. 14 schematically shows the user interface 112 with the selected compliance visualizations from FIG. 13. This user interface is visible to the medical practitioner when they are looking at a given patient on the generated protocol. As shown, the display automatically populates the various selected visualizations selected at step 608 (e.g., RR, SpO2, FiO2, and HR).

Additionally, the user interface 112 may have a variety of additional added features. For example, the user interface 112 may display a protocol start indication 899 indicating the start time of the protocol relative to the displayed data. In a similar manner, illustrative embodiments may display a protocol end indication 900 indicating the end time of the protocol relative to the display data. The user interface 112 may include a user-selectable marker 901 adjacent to the protocol start indication 899. Furthermore, the user interface 112 may be configured to indicate non-compliant periods 140 (e.g., represented by shaded areas of the patient data).

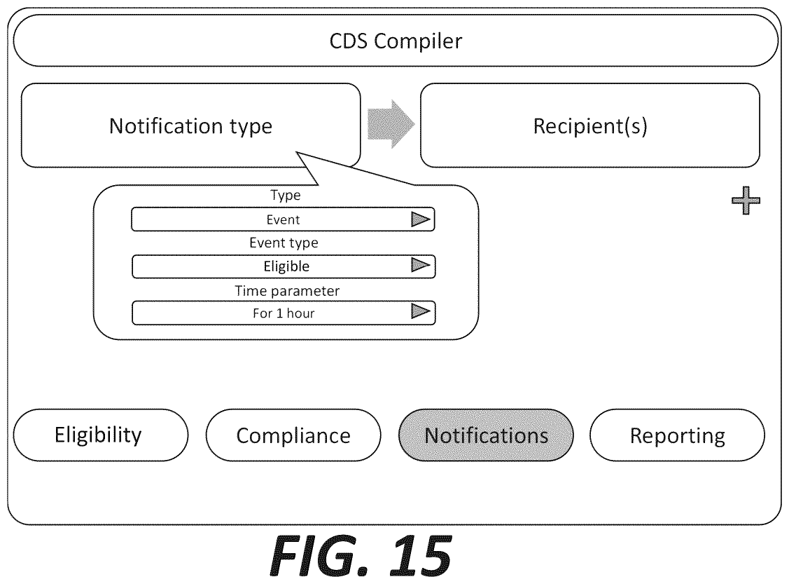

At step 610, the process sets the notification conditions. To that end, the user may configure the notification rule 130 within the interface 112. FIGS. 15-17 schematically show the user interface 112 for configuring the notification rule 130 in accordance with illustrative embodiments. From the user interface 112, the user selects the “notifications” menu item. The type of notification may be set. The notification may include details about the type of notification (e.g., event based, parameter based, etc.) and for the amount of time since the triggering of the notification (e.g., instantly, 1 hour, etc.). For example, the user may select to provide a notification when the patient is eligible for the protocol for 1 hour. Of course, the notification may relate to any of the various conditions 115 or rules for the protocol 120.

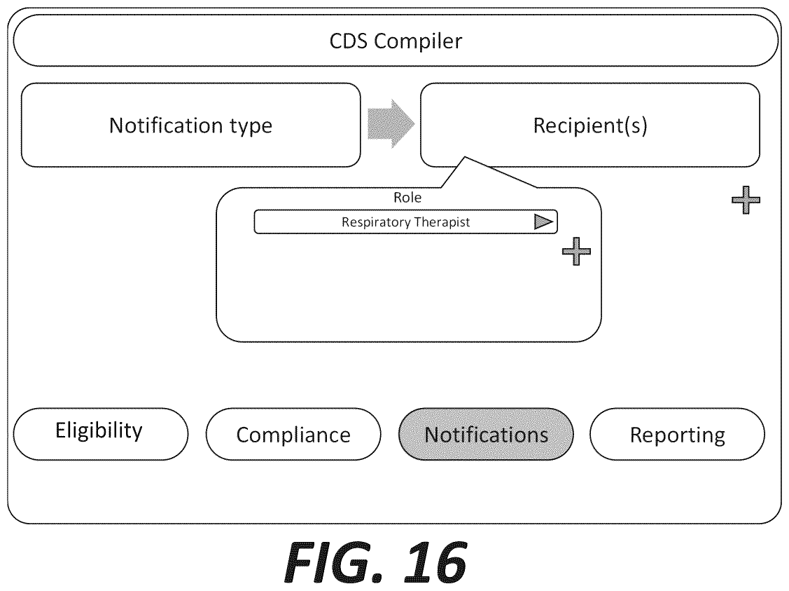

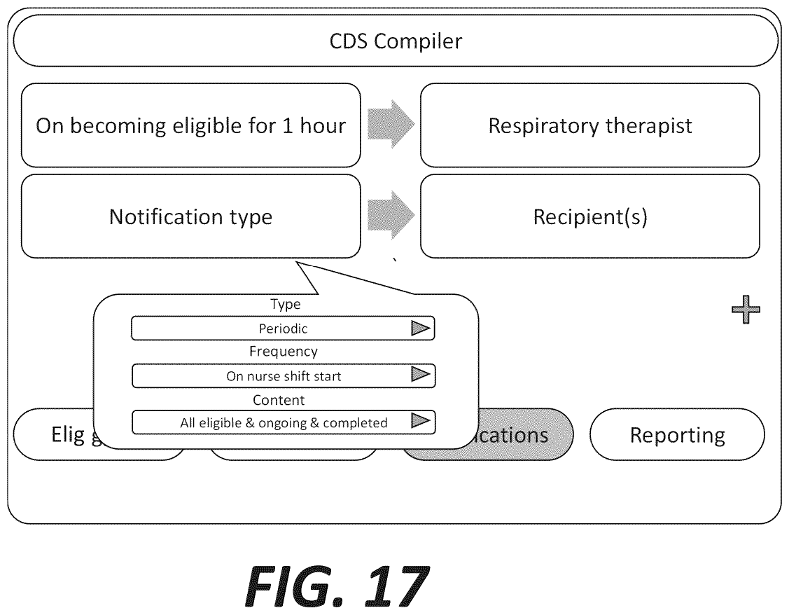

As shown in FIG. 16, the recipients who receive the notification may also be selected. In various embodiments, the associated medical practitioners may be pre-populated within the recipient field. For example, the respiratory therapist may be elected to be notified when the patient is eligible for 1 hour. As shown in FIG. 17, multiple notifications may be set. Additionally, various embodiments may choose to make one or more of the notifications a BPA (e.g., by sending to notification and/or notification requirements to the EHR 103 via the FIHR interface 107).

At step 612, the process 600 sets the reporting condition for the report generated by the quality reporting module 114 (e.g., based on real-time data or data from the simulator 142). While the notification conditions are intended to keep medical practitioners updated in real-time regarding patient specific actionable insight, the reporting conditions are intended to provide clinical setting (e.g., for the entire hospital unit) actionable insight. In other words, the reporting conditions report on the performance of the medical staff relative to the protocol 120. For example, the report condition may include the average time it takes for the medical staff to initiate the course of action from the time that the patient is eligible for the course of action. Additionally, or alternatively, the report conditions may include other associated action items, treatments, and/or courses of actions associated with the competition of a clinical protocol or the accomplishment of a given clinical outcome (e.g., parameter at a particular value, change in patient status, etc.).

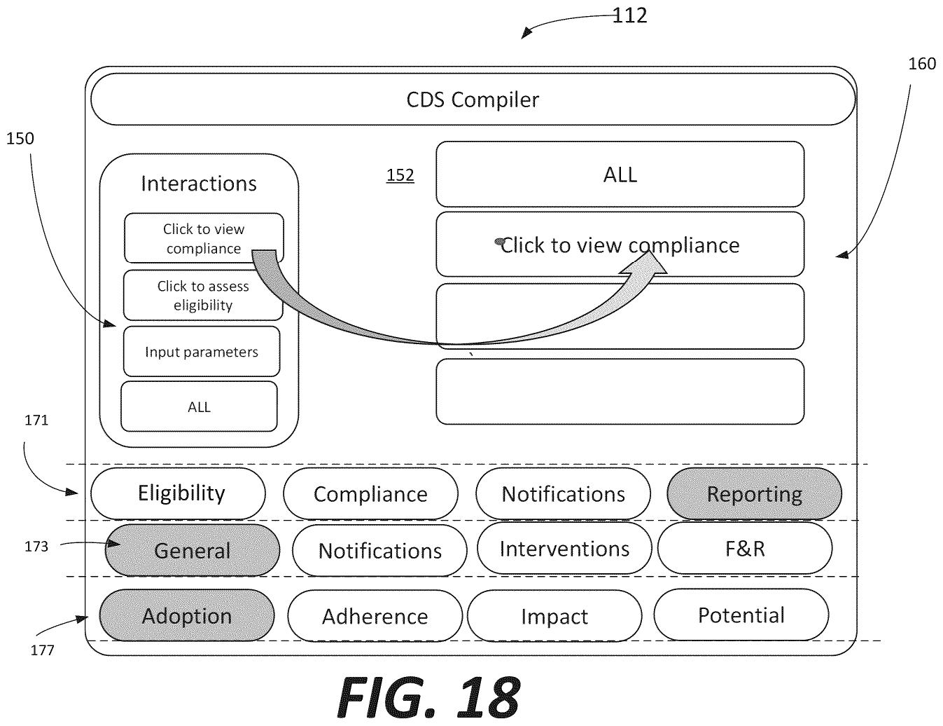

FIGS. 18-22 schematically show generation of reporting conditions using the user interface of the graphical compiler in accordance with illustrative embodiments of the invention. In various embodiments, the reporting module 114 includes a reporting sub-menu 177. In this embodiment, the reporting sub-menu 177 includes reports for “adoption”, “adherence”, “impact”, and “potential”. The reporting sub-menu 177 may include additional or fewer menu items. The user interface 112 is not intended to limit various embodiments.

FIG. 18 schematically shows the configuration user interface 112 for the reporting module 114 in accordance with illustrative embodiments. As discussed previously, the active user interface 112 provided to the medical practitioner when tracking patients may have a number of different views that the medical practitioner may alternate between (e.g., a census view (as shown in FIG. 3), a protocol view (as shown in FIG. 14), etc.). Illustrative embodiments may track how often and/or how many times the medical practitioner interacts with individual protocols (e.g., clicking on eligibility flag and going to main screen, changing a parameter of the protocol, etc.). Thus, illustrative embodiments may track user selections (e.g., touch screen selections, mouse inputs, etc.), among other things.

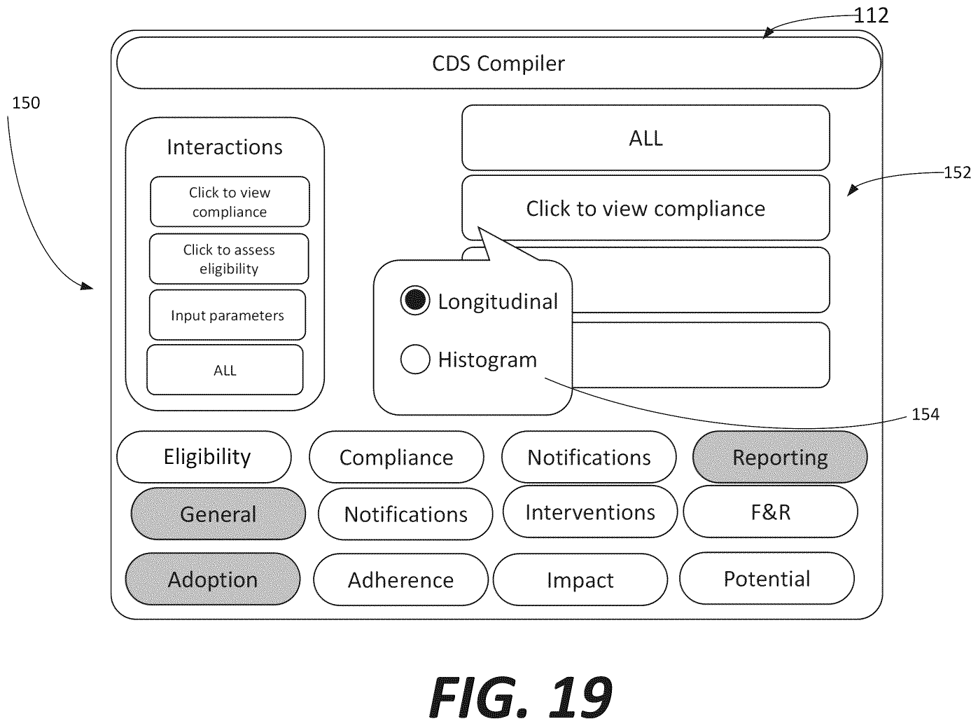

FIG. 18 schematically shows two selected graphical objects 160. In this instance, the reporting conditions that are being tracked are “all” interactions (i.e., the total number of clicks in the interface). The user then selects the “clicks to view compliance” graphical object 150, which is added as a reporting condition in the sequence window 152. FIG. 19 schematically shows the configuration user interface 112 of FIG. 18 after selection of the “clicks to view compliance” graphical object 150. In this instance, the graphical object offers to display the reporting as a longitudinal chart or a histogram.

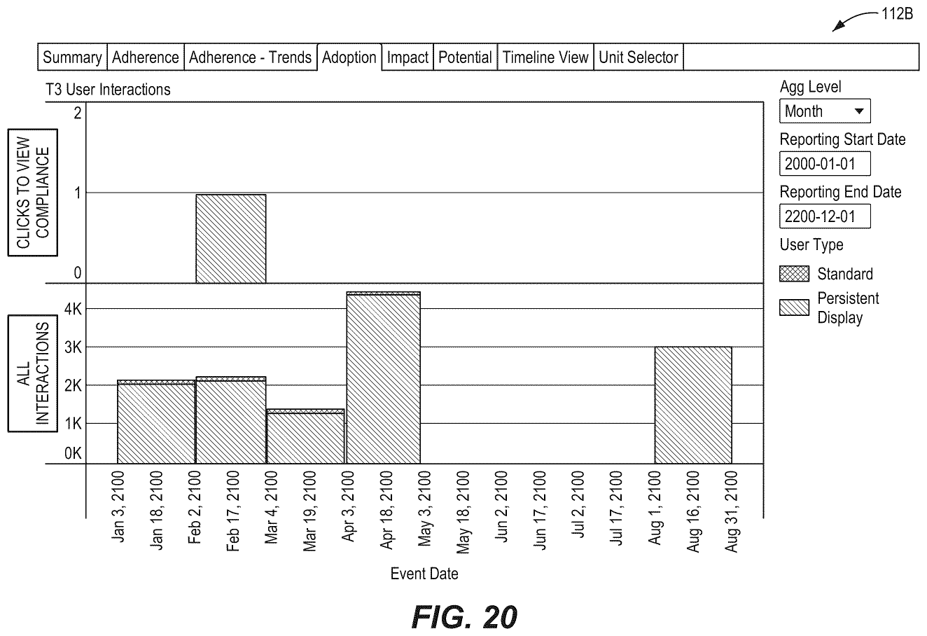

At step 613, the process generates a report. FIG. 20 schematically shows a report generated based on the selections in FIGS. 18-19. The report may be viewed in a report interface 112B (e.g., by a hospital administrator). For the sake of clarity, the report interface 112 may be, but is not necessarily, part of the same interface as the active interface 112A and/or the configuration interface 112. Furthermore, the report interface 112B may be, but is not necessarily, viewed on the same device as the active interface 112A and/or the configuration interface 112. For simplicity purposes, the three interfaces are described herein as being part of the same interface 112 (e.g., on the same display device). However, in various embodiments these interfaces 112A, 112B, and 112 may be separate (e.g., viewed on different displays by different users).

As shown, the report indicates the number of “all interactions” as well as the number of “clicks to view compliance.” In this way, the report provides insight regarding how medical practitioners interact with the active user interface 112.

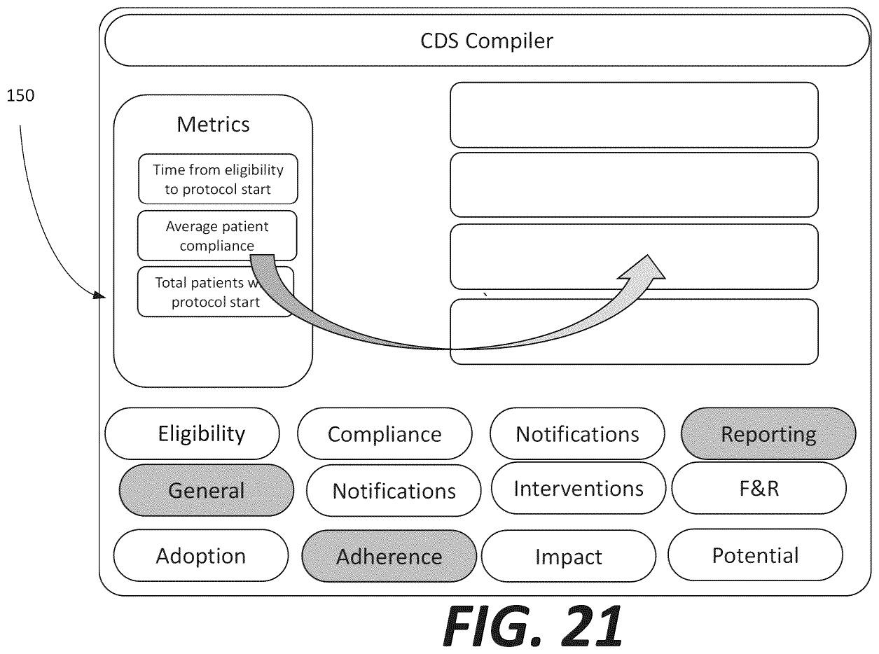

FIG. 21 schematically shows setting reporting conditions using the user interface of the graphical compiler in accordance with illustrative embodiments of the invention (e.g., step 612). FIG. 21 is similar to FIG. 18, except that the “adherence” button has been selected under the reporting sub-menu option. As shown, by switching to different sub menu-items, the interface 112 changes. In particular, the selectable graphical objects 150 change. In this case, a number of graphical objects relating to medical practitioner adherence with the protocol are populated. For example, the user may choose to track and display “time from eligibility to protocol start” in the report. As another example, the user may choose “average patient compliance” (e.g., 80%).

Some embodiments may include a large list of selectable graphical objects 150. However, some embodiments may use the sub-menu to assist with easy identification and management of relevant selectable graphical objects. Various embodiments may include the “adherence” sub-menu item. Adherence selectable graphical objects relate to reports about the performance of the medical practitioner/user (as opposed to the patient). For example, adherence selectable graphical objects 150 may include options such as: average wait time every month from patient eligibility to initiating the course of action for the protocol. This is a medical staff metric, as the patient does not control when they are placed on the course of action after they become eligible.

As an example, if medical staff strictly adhere to the protocol, every time a patient is flagged as eligible (e.g., for an ERT protocol), they instantaneously initiate the course of action (e.g., switch the ventilator mode to see how well the patient is breathing). Adherence allows the system 100 to track statistics such as the average time from patient being flagged to actually initiating the course of action (i.e., the patient has begun the protocol).

In some embodiments, adherence may be measured over time. For example, the system 100 may how many minutes until medical staff put patient on the protocol. Advantageously, the total time that elapses between the patient being eligible and being put on the course of action can be used to help direct resources and/or training. For example, if it takes medical staff 6 hours on average before the put the patient onto a protocol, it is possible that the patient may have kidney injury or neurological damage, etc. Thus, hospitals can track metrics that may be used to adherence to the protocol from the medical staff perspective.

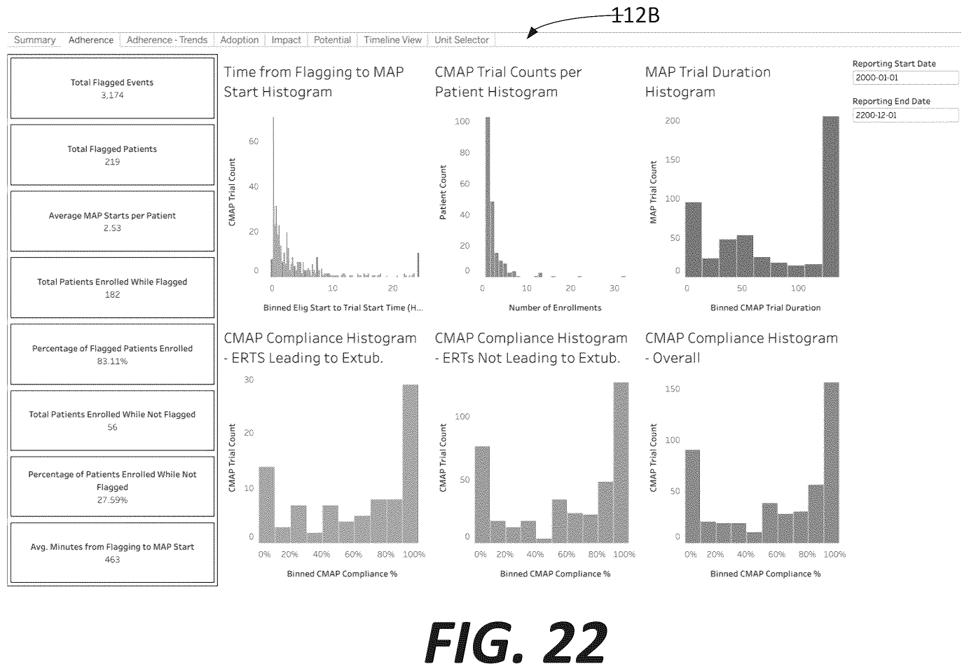

FIG. 22 schematically shows another example of a report generated based on the selections in the adherence sub-menu interface (step 613). The report may be viewed in the report interface 112B (e.g., by a hospital administrator).

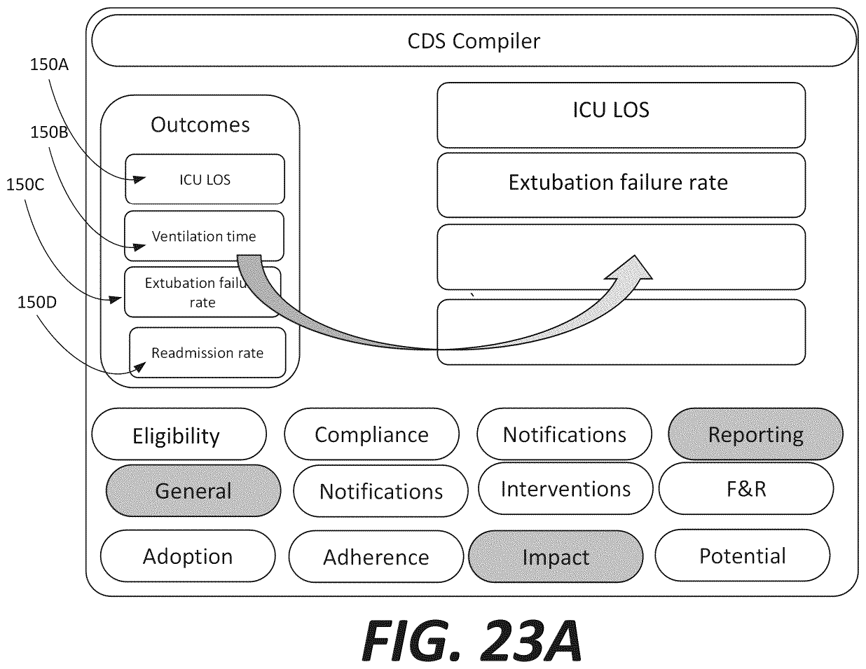

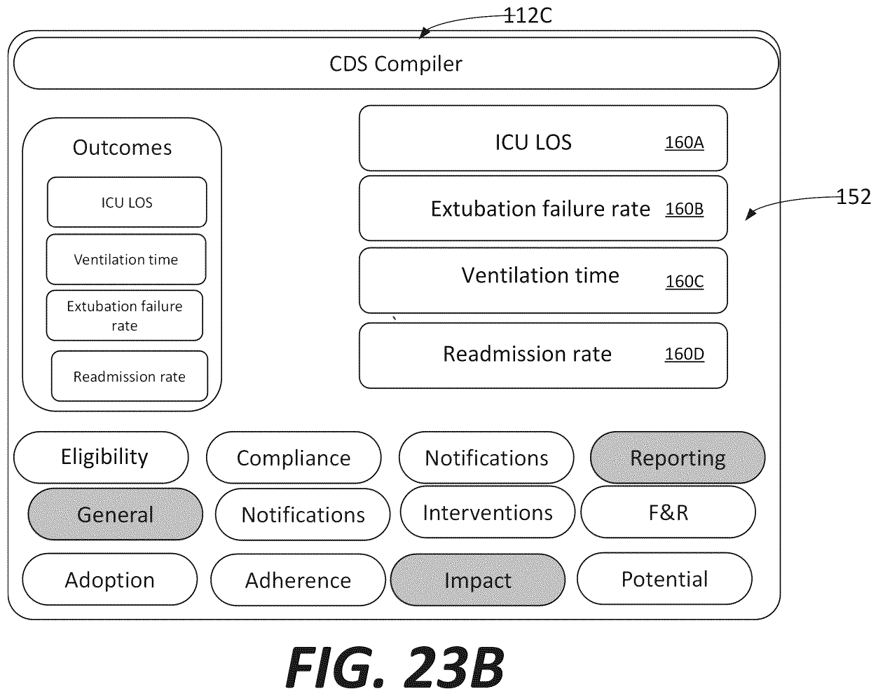

FIG. 23A schematically shows another example of setting reporting conditions using the user interface of the graphical compiler in accordance with illustrative embodiments of the invention (step 612). FIG. 23A is similar to FIGS. 18 and 21 except that the “impact” button has been selected under the reporting sub-menu. As shown, selecting a different sub menu-item causes the interface 112 to change. In particular, the selectable graphical objects 150 change. In this case, a number of graphical objects relating to protocol impact are populated. For example, the user may choose to track and display “time from eligibility to protocol start” in the report. As another example, the user may choose “average patient compliance” (e.g., 80%). FIG. 23B shows the interface of FIG. 23A after the user has selected graphical objects. The selected four graphical objects 160A-160D are populated in the sequence window 152.

In various embodiments, the user may choose a particular patient outcome (e.g., ICU length of stay, time on ventilation, deaths, extubation failure rate, etc.). The chosen outcome(s) are longitudinally shown over a selected timeframe. For example, time on ventilation per month is shown. Advantageously, this allows the hospital to see if the medical staff are adopting the policies correctly and the impact of that adherence.

The selectable graphical objects under the impact tab may relate to a particular patient outcome (e.g., ICU length of stay, time on ventilation, deaths, extubation failure rate, etc.). These graphical objects are used to provide a report that shows the particular outcome is over time (e.g., every month). For example, time on ventilation may be shown every month. Advantageously, these metrics allow the hospital to see if medical staff are adopting the protocol policies correctly and what the impact is.

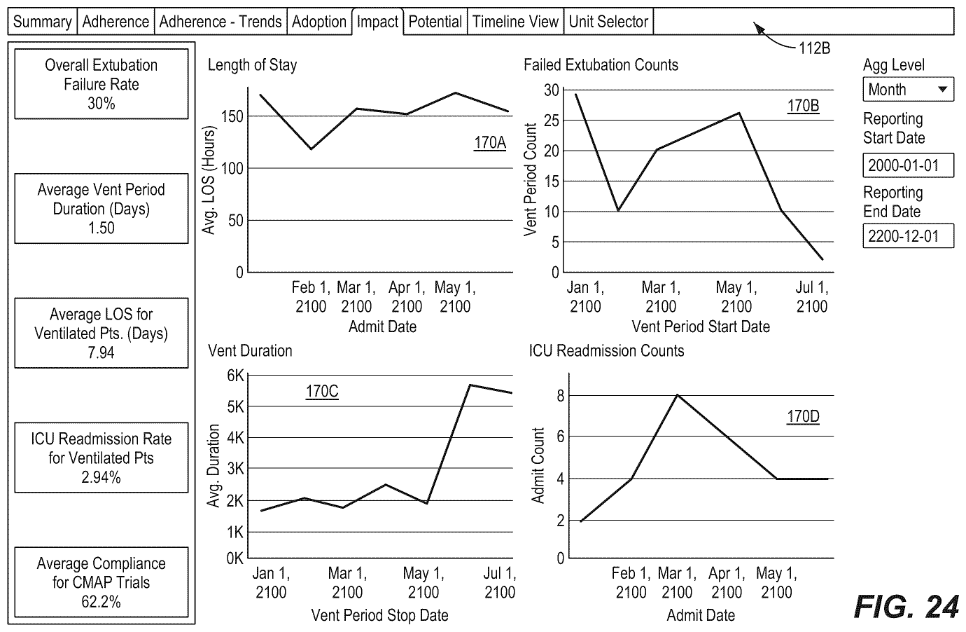

FIG. 24 schematically shows another example of a report generated based on the selected graphical objects 160A-160D from FIG. 23B. The report may be viewed in the report interface 112B (e.g., by a hospital administrator). As shown, the patient length of stay 170A, the failed extubation counts 170B, the vent duration 170C, and the ICU readmission counts 170D are displayed in the report, as selected by the user in the configuration interface 112C.

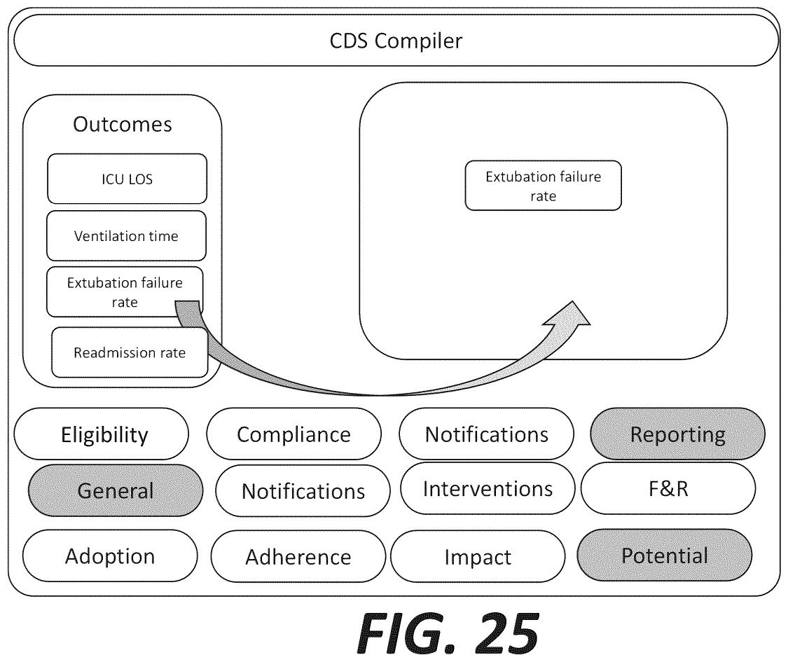

FIG. 25 schematically shows the user interface of the graphical compiler in accordance with illustrative embodiments of the invention. FIG. 25 is similar to FIG. 18, except that the “potential” button has been selected under the reporting sub-menu option. As shown, by switching to different sub menu-items, the interface 112 changes. In particular, the selectable graphical objects 150 change. In this case, a number of graphical objects relating to potential outcomes for the protocol are populated. For example, the user may choose to track and display “extubation failure rate” in the report. As another example, the user may choose “ventilation time” (e.g., 3 days 15 hours 20 seconds).

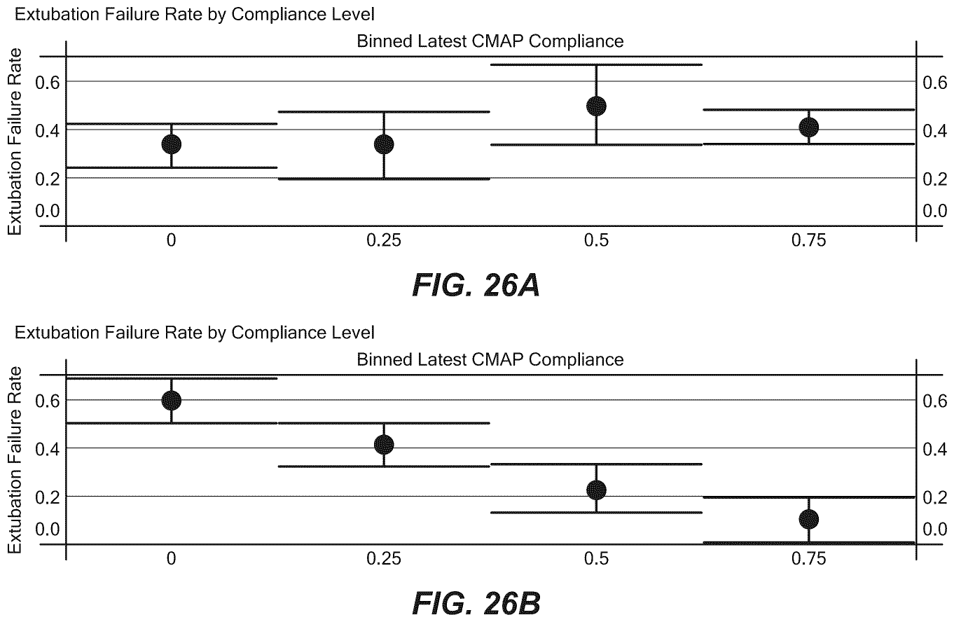

Some embodiments may include a large list of selectable graphical objects 150. However, some embodiments may use the sub-menu to assist with easy identification and management of relevant selectable graphical objects. Various embodiments may include the “potential” sub-menu item. Potential selectable graphical objects 150 relate to reports relating to whether the protocol has the potential to improve patient outcomes. The potential reports (e.g., shown in FIG. 26A-26B) show difference sin outcomes given different compliance rates for the patients.

FIG. 26A schematically shows the extubation failure rate report for a first protocol in accordance with illustrative embodiments. The extubation failure rate is mapped in accordance with patient compliance quartile (i.e., 0%-25% compliant, 26%-50% compliant, 51%-75% compliant, and 76%-100% compliant). As shown in FIG. 26A, there appears to be little to no correlation between patient compliance rate with the protocol and extubation failure rate. Accordingly, these results indicate to the medical practitioner that the current compliance conditions for the protocol do not have the potential to positively impact extubation failure rate. Thus, the process may proceed to step 614, and the compliance conditions may be adjusted.

FIG. 26B schematically shows the extubation failure rate report for a second protocol in accordance with illustrative embodiments. The second protocol may be a slightly modified version of the first protocol. For example, certain compliance parameters may be adjusted, added, and/or removed. By looking at the extubation failure rate by compliance level, the potential for the protocol becomes very clear. There is an approximately linear relationship between extubation failure rate and compliance level. Thus, hospital staff know that compliance with this protocol results in a desirable clinical outcome. Accordingly, hospital staff may prioritize maximizing patient compliance with the protocol because the potential for patient clinical success on the protocol is easily visualized.

In various embodiments, a theoretically ideal protocol should have the highest positive clinical outcome (e.g., lowest extubation failure rate) for the most compliant patients. The reporting shows the medical practitioners that compliance with the protocol used for FIG. 26B makes a significant difference. Also of significance to the medical staff, the reporting indicates that the current medical protocol used for FIG. 26A does not have a positive impact on patient outcomes. Thus, medical staff have actionable insight that they may use to determine (or adjust) an appropriate protocol. Illustrative embodiments thus provide actionable insight by measuring potential.

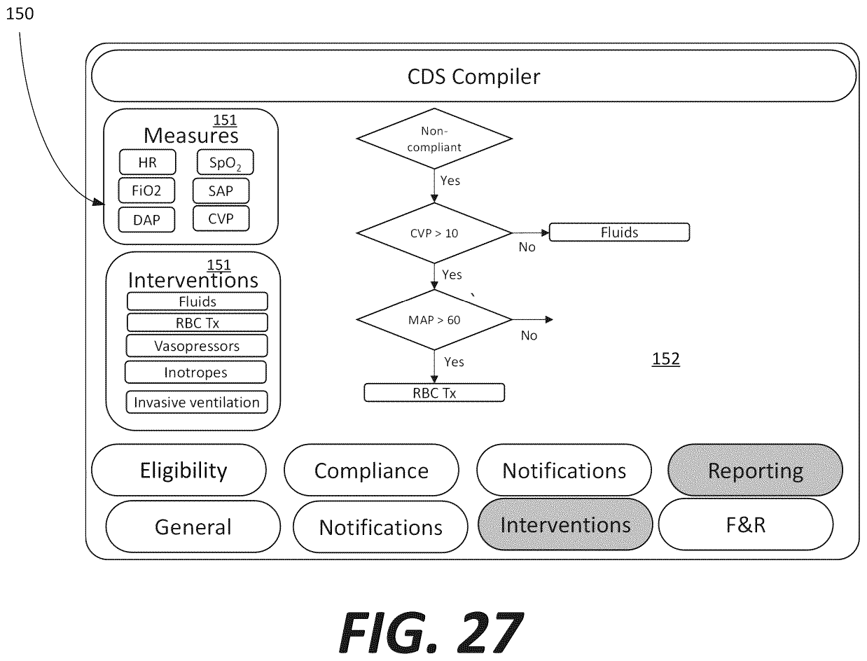

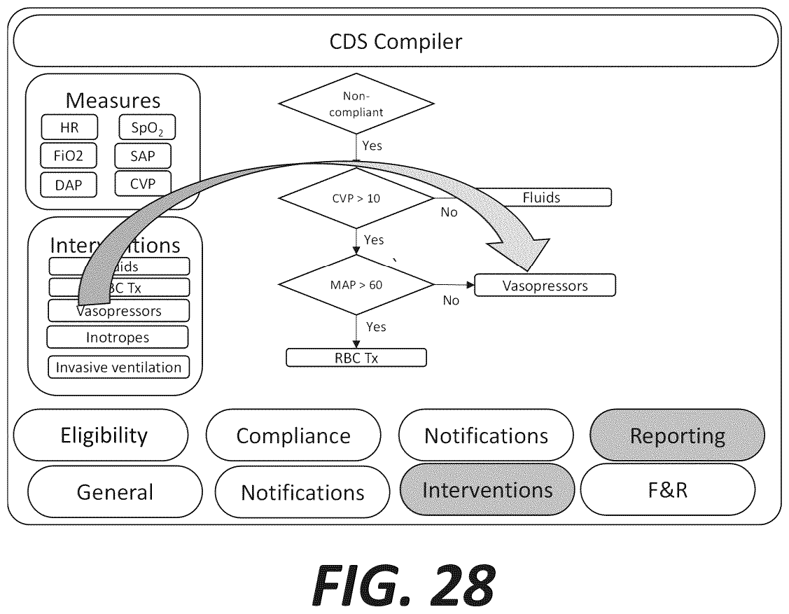

The reporting conditions may also include interventions that occur when the patient is non-compliant. For example, FIGS. 27-28 schematically show the user interface 112 for configuring the reports in accordance with illustrative embodiments.

The reports generated by the system 100 provide actionable insight and objective analysis of the performance of the medical staff in relation to eligibility and compliance criteria. For example, among other things, the quality reporting module 114 tracks the time elapsed from patient eligibility until a user acknowledges patient eligibility (e.g., medical practitioner interacts with notification). Alternatively, or additionally, the quality reporting module 114 may track the time elapsed from patient eligibility until a medical practitioner begins the course of action associated with the eligible protocol for the patient 101. Alternatively, or additionally, the time elapsed from non-compliance to an associated patient intervention may be tracked. By tracking this information, medical practitioner performance may be objectively measured (e.g., which medical practitioners are the fastest responders and what are the impacts of response time on patient 101 clinical outcomes for given protocols).

General reporting protocols may be setup through the configuration interface 112. Additionally, the specific interventions may be established through the reporting interface. Thus, the graphic compiler 146 allows the medical practitioner to set the expected interventions for medical practitioners when a patient is non-compliant. FIG. 27 schematically shows the configuration interface 112 for creating reporting associated with the protocol 120.

Users may generate the decision tree using similar methods to those as described above. An object library 151 (e.g., measurement library and/or intervention library) is pre-populated with a plurality of graphical objects. Selection of the graphical objects 150 moves those objects into the selected objects window 152.

The interface 112 also allows the user to build out the decision tree for reporting. For example, when the patient is non-compliant, and CVP>10, then the intervention is to give fluids. The reporting module may track how long it takes from non-compliance and CVP>10 until fluids are provided.

FIG. 28 schematically shows an example of the user building the decision tree. In the same decision tree as FIG. 27, if CVP>10, but MAP>60, the intervention is red blood cell transfusion. However, if MAP>60, the user may indicate that the intervention should be to provide the patient with vasopressors. The quality reporting module 114 may thus track the reporting as outlined by the user.

The quality reporting module 114 looks at the established protocol rules and provides a number of statistics. For example, what is the average time between the patient becoming non-compliant and applying any of the possible interventions. Additionally, the reporting module 114 may work with the simulator to determine the percentage of the time that the intervention is the correct intervention (e.g., net positive outcome based on historic data). Thus, the reporting module is advantageous from both a simulation perspective, but also during deployment. The reporting module 114 tells the user how well the clinicians are executing this particular protocol based on the reporting configurations set using the compiler 146.

The reporting may be configured to provide a number of different statistics, such as average times and histogram of times between patient becoming non-compliant and any possible intervention, the percentage that the desired intervention was applied, the intervention success rate at bringing the patient in compliance, etc.

The quality reporting module 114 may track a dynamic concordance rate as a percentage of time over which the patient 101 is compliant with the medical protocol. The quality reporting module 114 may also track the patient eligibility time. Additionally, the quality reporting module 114 may retrospectively look at patient concordance rate and/or eligibility time to see how well a particular patient and/or patients of a particular medical practitioner have performed relative to a patient population. Additionally, the reporting module 114 may be configured to display the received patient data and to mark non-compliant portions of the received data. Thus, the quality reporting module 114 allows medical facilities and/or staff to evaluate how well patients were managed retrospectively. For example, if eligibility times are very high, medical practitioners become aware of it. This assists with hospital policy formation and also with ensuring prompt action by the medical staff.

In illustrative embodiments, the quality reporting module 114 tracks the time at which the patient 101 became eligible for the protocol 120, and the duration from the eligibility time to a predetermined event. The predetermined event may be, for example, when a medical practitioner acknowledges patient eligibility (e.g., popup notification on touch screen display via user interface 112), or when the course of action for the eligible protocol is initiated.

Illustrative embodiments may provide periodic reporting to assist medical practitioners with understanding facility wide compliance.

Returning to FIG. 6, the process proceeds to step 614, which asks if the protocol should be adjusted. For example, if a simulation was performed, and the quality reporting module 114 indicates that clinician performance is sub-par, that patient outcomes for high-compliance patients are not beneficial, or that the protocol results in too many notifications (e.g., alarm fatigue) the process may return to any and/or all of steps 602-612, and the protocol may be adjusted. This process may be iterated in real time by the medical practitioner as many times as desired. If the protocol does not need to be adjusted, the process proceeds to step 616, and the protocol is compiled and deployed to system. The process then comes to an end.

It should be apparent to one skilled in the art that the process of FIG. 6 may be incorporated into the process of FIG. 5. Particularly, the process 600 shows a detailed view of step generating the protocol received in step 502. Thus, the protocol generated using the process of FIG. 6 may be used in the process outlined in FIG. 5. Furthermore, at step 512, the graphical compilation method described above with respect to the process 600 may be used to modify the protocol or create a new protocol.

It should further be understood that various steps may be modified or omitted in illustrative embodiments. For example, some embodiments may skip step 612, and may not include reporting conditions associated with the protocol. In some embodiments, the reporting conditions may be related to one or more protocols.

Furthermore, in some embodiments, the step of deploying the protocol 616 made additionally, or alternatively, come at the beginning of the process. For example, the method 600 of FIG. 6 may be used to modify and/or analyze a previously deployed protocol.

In various embodiments, the reporting module 114 communicates with the analyzer 144. The analyzer 144 is configured to analyze the patient data and extract outcomes from the data to assist with reporting. The outcomes may include, among other things, ICU length of stay, ventilation time, etc. To that end, the analyzer 144 includes an event detector 149 configured to scrub the data from the sensors 102 and medical devices 104 and automatically determine these outcomes without the need for manual input from medical staff. However, in some embodiments, the database 105 may provide the outcomes directly or the outcomes can be extracted as a secondary interaction with the patient database 105.

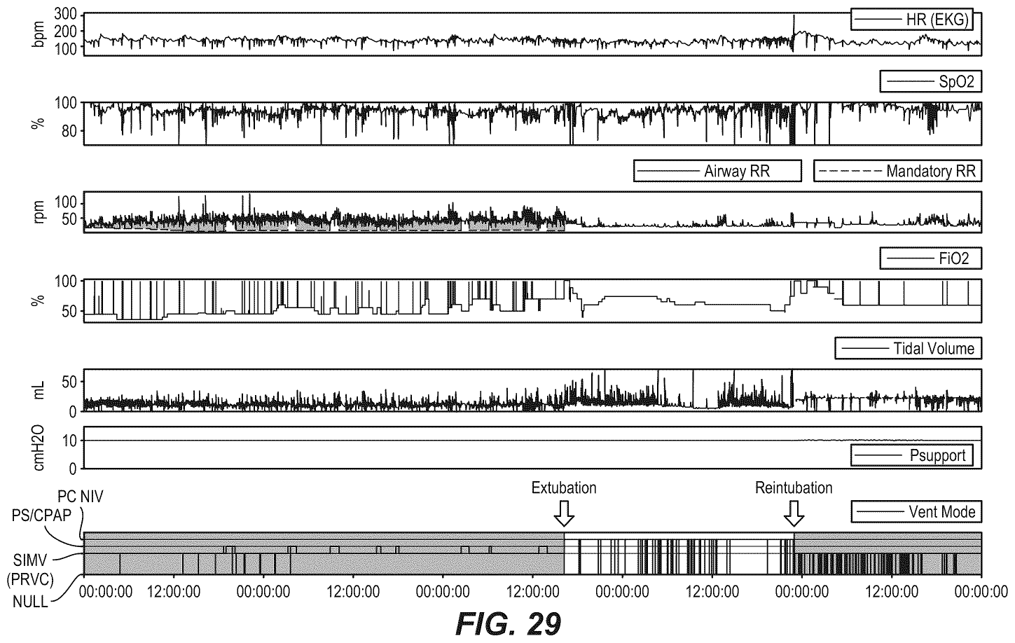

FIGS. 29-32 schematically shows views of data collection that may be used by the analyzer 144 to automatically determine clinical events and/or outcomes.

FIG. 29 schematically shows a view of the protocol interface 112 in accordance with illustrative embodiments. The analyzer 144 is configured to detect, among other things, when the patient is extubated and intubated. FIG. 29 schematically shows ventilated data. The ventilator data (bottom-most chart) shows collected ventilator data which then stops collecting. The analyzer 144 is configured to determine the mode of the ventilator from the patient data (including the ventilator data). In this example, the data line (i.e., blue line) indicates the ventilator mode. At one point (green arrow labeled “extubation”), the blue line shifts up to PC NIV. That is a non-invasive ventilation mode. This indicates that the ventilator breathing tube is removed from the patient. As shown in the figure, the subsequent period of time there may include some noise in the data signal, but at some point (e.g., identified by the red arrow labeled “reintubation”) the ventilator data returns into an SIMV mode, which is an invasive ventilation mode that requires intubation. Above that is Pressure Support, when a patient is no longer intubated the sensors 102 are unable to measure pressure support anymore. When the ventilation mode switches, the event detector 149 may automatically determine that the patient was extubated. Then, at a later time, the data reappears and the event detector 149 can determine that the patient was reintubated.

Between the extubation and reintubation, the ventilator switches from non-invasive mode to null, which means that the ventilator is not working/not collecting data. This is a standard data plot that may be obtained from a ventilator. The event detector 149 scrubs this data and determines that the patient was extubated.

As shown below, the event detector 149 may be configured to detect a variety of clinically significant events (e.g., when a patient develops acute kidney injury by the fact that their creatinine jumps, that the patient has cardiac arrest from heart rate going, etc.). Illustrative embodiments extract outcomes/events using machine learning/pattern recognition from the patient data. Furthermore, these outcomes/events may be used as conditions 115 in any of the rules.

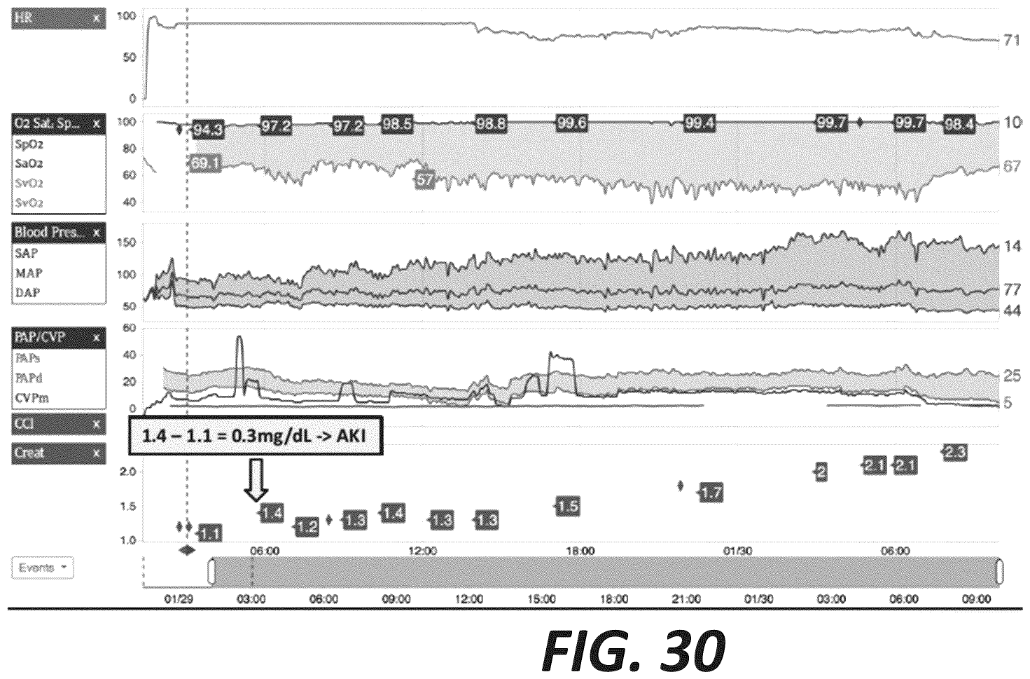

FIG. 30 schematically shows a view of the protocol interface 112 in accordance with illustrative embodiments. The event detector 149 may automatically detect, among other things, acute kidney injury (AKI). For patients who have a normal creatinine level (e.g., adult men: 0.74 to 1.35 mg/dL adult woman: 0.59 to 1.04 mg/dL) at ICU admission, the first time their creatinine increases more than 0.3 mg/dL within 48 hours during their ICU stay is defined as acute kidney injury (AKI).

The event detector 149 parses the data and determines that the patient's first creatinine level is normal after ICU admission. Then, the event detector 149 determines that the creatinine increased more than 3 mg/dL within 48 hours during their ICU, and that this is the first time this increase has happened. The analyzer 144 may evaluate the patient data for current creatinine lab result minus the minimum preceding creatinine level, to determine that the patient creatinine has increased more than 0.3 mg/dL. Thus, the event detector 149 determines the time when the current creatinine lab was taken as the time of the event (e.g., AKI) and/or that the patient has a specific condition/outcome (e.g., AKI). The event detector 149 therefore determines events, conditions, and/or outcomes from the patient data, as well as the time of onset, duration, and or ending.

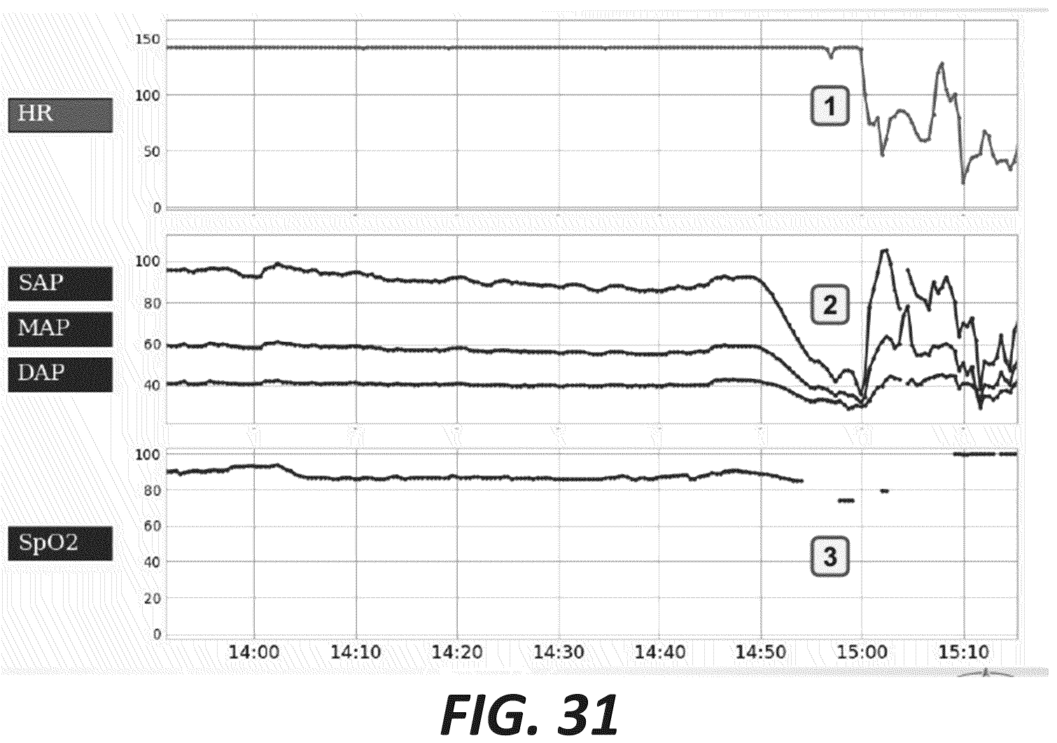

FIG. 31 schematically shows a view of the protocol interface 112 in accordance with illustrative embodiments. The data in this example is used by the event detector 149 to determine when the patient has cardiac arrest. Cardiac arrests can have a variety of signatures in a patient's vitals, but most commonly exhibit:
1. A precipitous drop in heart rate followed by large variations
2. A drop in blood pressure as well as a narrowing in the difference between systolic and diastolic pressures
3. A drop and/or disappearance of the peripheral oxygen saturation (SpO2).

By analyzing the data and looking for these specific signatures, the event detector 149 may determine when the patient has cardiac arrest. The three conditions that the event detector 149 searches for are labeled in FIG. 31. Accordingly, the event detector 149 determines that the patient has cardiac arrest, and the time at which the cardiac arrest occurred.

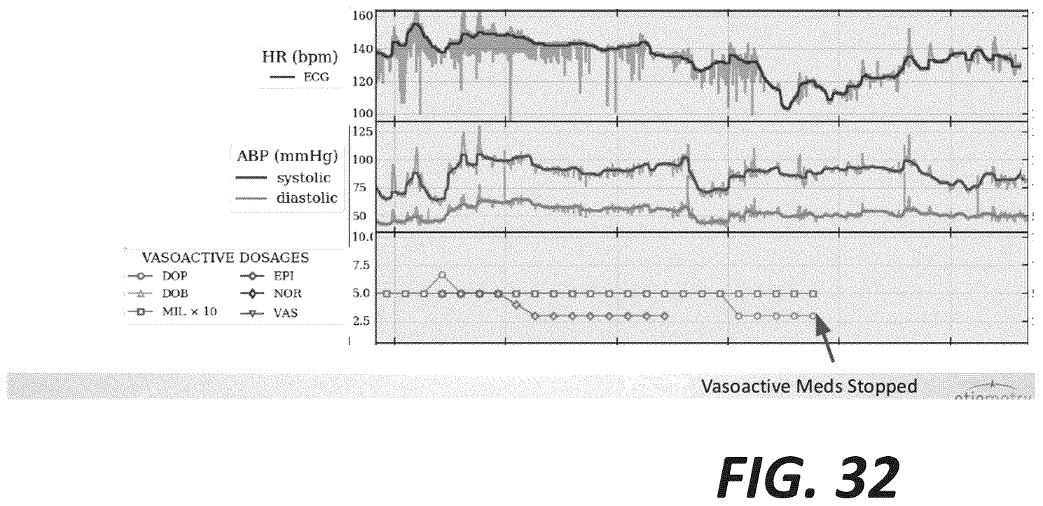

FIG. 32 schematically shows a view of the protocol interface 112 in accordance with illustrative embodiments. In this example, the event detector 149 is configured to detect, among other things, when the patient vasoactive weans. Vasoactive weans are defined as the complete cessation of all infusions of the six vasoactive medications:
Dopamine
Dobutamine
Milrinone
Epinephrine
Norepinephrine
Vasopressin

As shown, the event detector 149 is configured to detect vasoactive weaning from the patient data as well as the time at which weaning begins.

It should be understood that similar to the other rules and reporting described herein, the event detector 149 may be configured to detect various events/conditions/outcomes using the graphical compiler. Additionally, or alternatively, the event detector 149 may be preconfigured to detect the various events/conditions/outcomes. Furthermore, results obtained by the event detector 149 may be used as conditions 115 in one or more rules.

It should be apparent to one skilled in the art that illustrative embodiments provide a number of advantages over the state of the art. As an initial matter, illustrative embodiments advantageously provide a system for the creation and modification of all aspects of a clinical protocol, from eligibility to reporting, using an intuitive graphical object based interface. Furthermore, illustrative embodiments provide for simulation of a protocol, such that the simulated protocol performance (e.g., patient dynamic concordance rate) may be used as a feedback mechanism to adjust the protocol (e.g., adjust the protocol conditions if simulated patient compliance is good but patient outcomes are bad, adjust the notification conditions if too many notifications, etc.). Furthermore, custom reports may be generated to determine medical staff performance (e.g., how quickly is medical staff acting, are they following the appropriate interventions, etc.). Thus, illustrative embodiments advantageously enable a complete review, and rapid revision, of the effectiveness of a medical protocol, from both the patient outcome perspective and/or the clinician execution perspective.

Additionally, various embodiments advantageously provide reporting that shows the overall performance of the medical practitioners/clinical setting. This advantageously allows hospitals and other clinical care settings to analyze medical staff metrics. Currently, health care has a lot of quality metrics. The quality metrics may be related to how the hospital gets reimbursed by the insurance company. Illustrative embodiments enable clinical environments to identify that a particular protocol was implemented, and the medical staff compliance rate, as well as the practical outcomes of implementing the protocol (e.g., reduced extubation failure rate from 15% to 10%).

Furthermore, the reporting module 114 provides specific reports related to, among other things, adoption and adherence by medical staff, as well as impact and potential of the protocol.

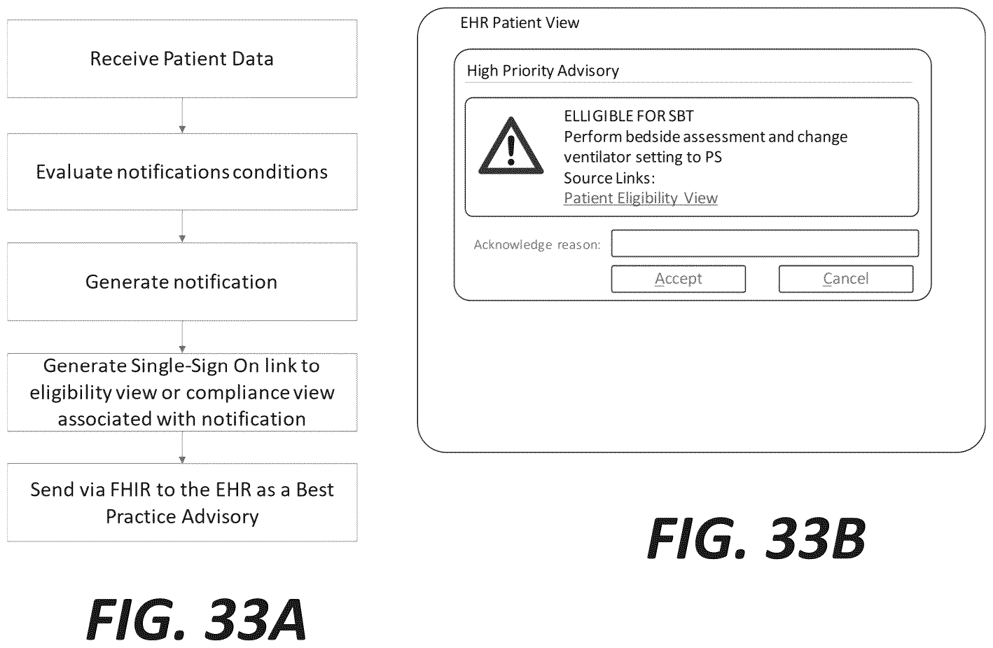

FIG. 33A shows a process of creating a best practice alert in accordance with illustrative embodiments. It should be noted that this method is substantially simplified from a longer process that may normally be used. Accordingly, the method shown in FIG. 33A may have many other steps that those skilled in the art likely would use. In addition, some of the steps may be performed in a different order than that shown, or at the same time. Furthermore, some of these steps may be optional in some embodiments. Accordingly, the process of FIG. 33A is merely exemplary of one process in accordance with illustrative embodiments of the invention. Those skilled in the art therefore can modify the process as appropriate.

As described previously, the system 100 receives patient data for a patient on a given protocol. The notification conditions for the protocol are evaluated by the system 100, and the system 100 generates a notification when the conditions are met. These steps have been described in more detail previously, and are not repeated here.

The notifications may be pushed through to the medical practitioner (or interested party) via the electronic health record 103 platform. Accordingly, illustrative embodiments may generate a single-sign on link that leads to the relevant view (e.g., eligibility view or compliance view) associated with the notification when selected. Furthermore, via the FHIR interface 107, the notification with the link may be sent to the EHR 103 as a best practice alert.

FIG. 33B shows a user interface of the electronic health record 103 in accordance with illustrative embodiments. In this particular example, the patient is eligible for an SBT protocol, and provides the next steps that should be performed to the medical practitioner (e.g., perform bedside assessment, etc.). The user interface 112 may be viewed via the EHR 103. For example, the EHR may show the generated single-sign on link which may be selected (in this example to view the patient census view (e.g., such as in FIG. 11) or an eligibility window, which shows relevant metrics relating to eligibility conditions for the particular patient).

It should be appreciated that accessing the system 100 via the EHR 103 interface provides a number of advantages. Medical practitioners spend a great deal of time in the EHR 103, and therefore, it is highly efficient from the perspective of the medical practitioner to integrate the system 100 into a user interface in the EHR 103 (e.g., via single sign on). This advantageously increases use of the system 100 and compliance with conditions of given protocols.

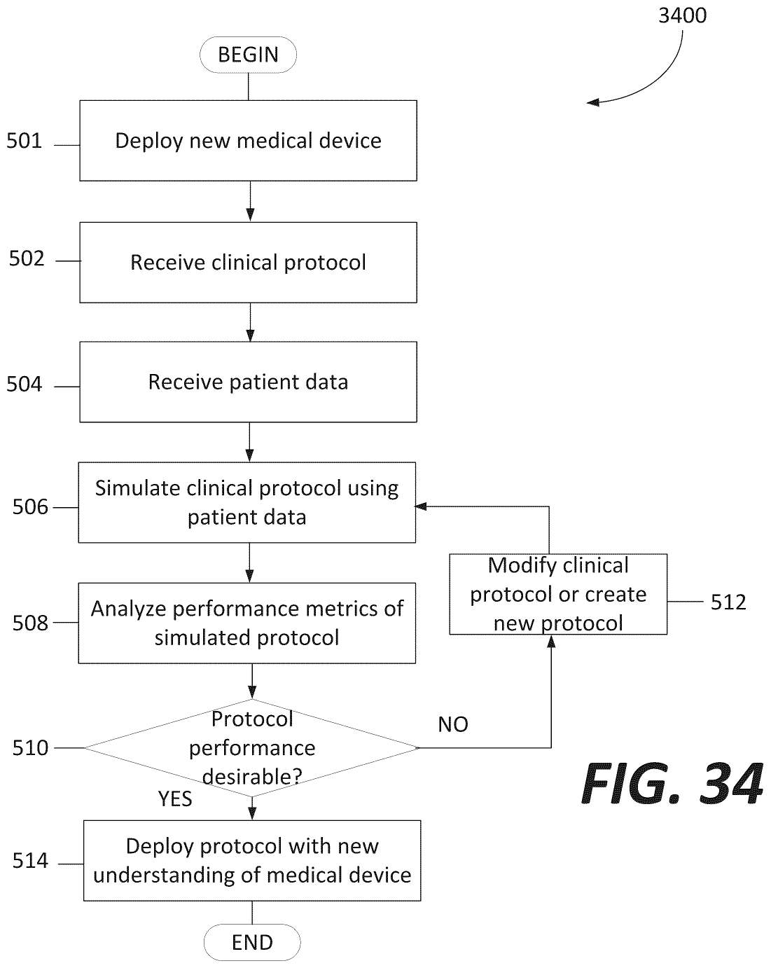

FIG. 34 schematically shows a process of generating and optimizing a protocol for newly deployed medical device in accordance with illustrative embodiments. It should be noted that this method is substantially simplified from a longer process that may normally be used. Accordingly, the method shown in FIG. 34 may have many other steps that those skilled in the art likely would use. In addition, some of the steps may be performed in a different order than that shown, or at the same time. Furthermore, some of these steps may be optional in some embodiments. Accordingly, the process of FIG. 34 is merely exemplary of one process in accordance with illustrative embodiments of the invention. Those skilled in the art therefore can modify the process as appropriate.

FIG. 34 is similar to the process shown in FIG. 5 and therefore contains many similar steps that are not again repeated in full detail. Advantageously, the process of FIG. 34 provides clinically relevant insights for new medical devices using the protocol generation and simulation methods described herein.

When a new medical device is available, it may be difficult to determine the patients that benefit from use of the device, the patient(s) that benefit the most from use of the device, particular beneficial parameters and/or dynamic concordance rates, and/or the best usage of the device in conjunction with other available devices and/or data. Various embodiments provide for flagging patients as eligible for use with a particular medical device (e.g., implantation of a heart pump) in a manner similar to how patients are flagged as eligible for a particular protocol (e.g., SBT). Additionally, when the new medical device is deployed to the patient for a given protocol, patient compliance can be managed using the system 100. Thus, patient eligibility, management, and compliance with protocols that use the new medical device may be determined and monitored through the system 100, and furthermore through the EHR 103.

The process of FIG. 34 begins at step 501, which deploys one or more new medical devices in a clinical setting. As an example, the new medical device may be a hemodynamic monitoring device used to optimize the management of cardiogenic shock. The new hemodynamic monitoring device may measure cardiac index.

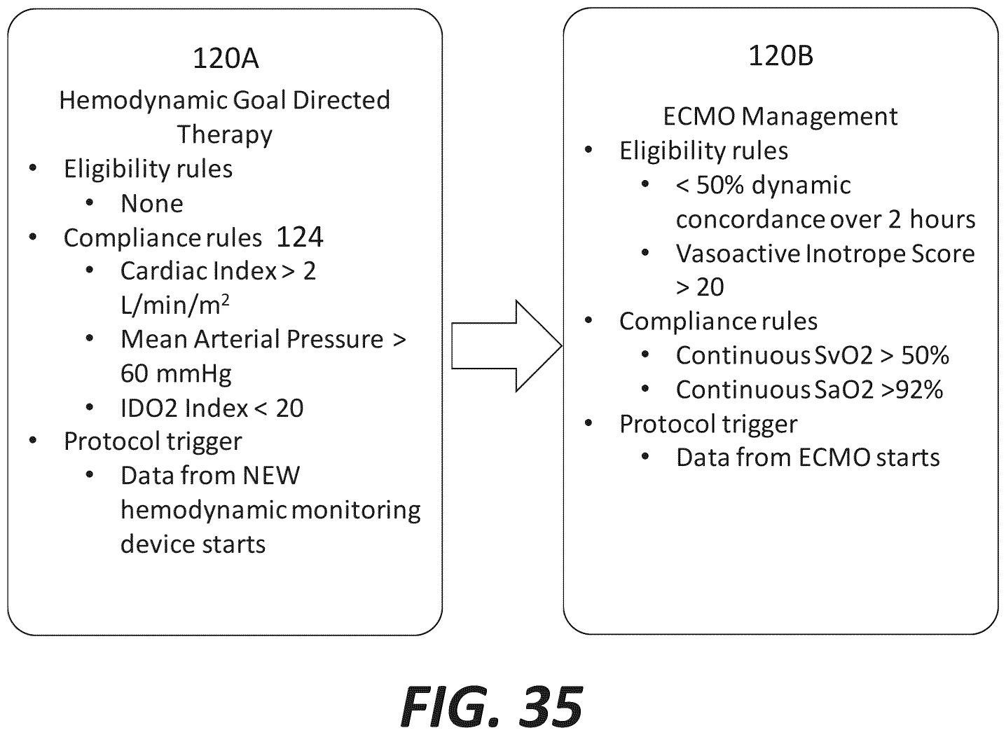

At step 502, the protocol 120 is received. FIG. 35 schematically shows an example of a protocol 120 that uses the new device. Of course, this protocol is merely exemplary, and a variety of different protocols may be used. In this example, the compliance rules for the particular protocol include a given measurement from the new device (e.g., cardiac index >2), as well as other measurements from other devices (e.g., mean arterial pressure and IDO2 index).

At step 504 patient data is received. The patient data may include historic patient data. However, the received patient data now includes data acquired from the new device (e.g., as well as the other data described with reference to FIG. 5). Advantageously, the system 100 enables the medical practitioner to determine how best to utilize the new collected data (e.g., cardiac index) together with the other collected data (e.g., mean arterial pressure) or data produced by the system using other collected information (e.g., IDO2 index from risk analytics engine). This is accomplished by generating a new protocol to manage the patient in a particular way.

At step 505, the protocol 120 is simulated and the dynamic concordance of patients on the protocol is tracked. In some embodiments, in the first instance of step 506, “simulating” the protocol may include actively running the protocol in real-time (e.g., in a clinical setting on real patients). At step 508, the performance metrics of the protocol are analyzed. By tracking the dynamic concordance, insights may be gained about the outcomes of using the new medical device. For example, analysis of the protocol may indicate that when used with the new medical device, a dynamic concordance of 95% or greater results in early discharge of patients from the hospital. Alternatively, no insight may be derivable, and the process may proceed to step 512 which modifies the protocol, and then determines the patient outcomes for the modified protocol.

By determining patient outcomes for the protocol using patient data, it is possible to establish insights that otherwise not known. For example, the analyzer may determine that patients have desirable clinical outcomes when their dynamic concordance is greater than 50% for over 2 hours on the protocol, particularly when vasoactive inotrope scope is greater than 20.

The process proceeds to step 510, which asks if the protocol has desirable performance. For example, if a desirable clinical outcome is seen with a given threshold % dynamic concordance, then the protocol performance is desirable. However, if no insight or desirable clinical outcome is gained from a given threshold % dynamic concordance, then the process proceeds to step 512, and the protocol is modified. The newly modified protocol may be simulated using the historic data collected previously at step 504. In this way, multiple different protocols may be simulated, and desirable outcomes may be determined as a function of a new medical device used with the patient.

The process then proceeds to step 514, which deploys the protocol with a new insight for the medical device. The new insight may include the patients that benefit from use of the device, the patient(s) that benefit the most from use of the device, particular beneficial parameters and/or dynamic concordance rates, and/or the best usage of the device in conjunction with other available devices and/or data. Multiple simulations may be run for multiple protocols using patient data from a variety of patients, and determinations may be made regarding best clinical outcomes for a variety of patients. Among other things, patient demographics, dynamic concordance, and protocol rules may all be optimized for clinical outcomes using the new medical device. The process then comes to an end.

Returning to the example shown in FIG. 35, the process of FIG. 34 may be used to determine, for example, that it is desirable to put patients on ECMO management protocol 120B when there is less than 50% dynamic concordance over 2 hours for the parent protocol 120A in combination with a vasoactive inotrope score of less than 20. In this example, the system 100 detects when hemodynamic targets cannot be achieved and flags the patient for escalation to mechanical circulatory support. (The Vasoactive Inotrope Score indicates the amount of support the patient is getting from medications. The higher the support is, the less likely is that more support leads to compliance with hemodynamic targets.)

The simulator enables the medical practitioner to choose the right combination of parameters (Dynamic concordance threshold over specific time and VIS score) to optimize the following outcomes:
Start mechanical circulatory support early enough to avoid end-organ injury from hypo-perfusion,
Avoid starting mechanical circulatory support too early given that this support is associated with significant morbidity, e.g., bleeding, stroke, etc., and
Improve overall survival from cardiogenic shock

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

As used in this specification and the claims, the singular forms “a,” “an,” and “the” refer to plural referents unless the context clearly dictates otherwise. For example, reference to “a rule” in the singular includes a plurality of rules, and reference to “the condition” in the singular includes one or more conditions and equivalents known to those skilled in the art. Thus, in various embodiments, any reference to the singular includes a plurality. In a similar manner, reference to more than one component (e.g., one or more, a plurality) can include the singular.

Also, as regards terminology, illustrative embodiments refer to the protocol as having “rules,” such as “eligibility rules” or “compliance rules”. Additionally, illustrative embodiments refer to each of the “rules” as having “conditions”. Earlier applications have referred to “conditions” and “rules” interchangeably (e.g., an eligibility rule may be referred to as an eligibility condition). However, for the sake of discussion, illustrative embodiments generally refer to a “rule” having “one or more conditions.” Furthermore, both the singular term “rule” and the plural term “rules” refer to one or more conditions. Thus, usage of the plural term “rules” (e.g., eligibility rules, compliance rules, subscription rules, notification rules, etc.) is meant to refer to the one or more conditions of the rule (e.g., the one or more conditions of the eligibility rule), and is not meant to imply that the protocol requires multiple different rules (e.g., multiple different eligibility rules).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A method of back testing a medical protocol comprising:
receiving an eligibility rule and compliance rule for a medical protocol, each of the rules having one or more conditions;
receiving collected patient data from a plurality of patients, the collected patient data including longitudinal patient data relating to a specific parameter collected over a period of time from one or more sensors and/or medical devices;
determining that a patient was eligible for the protocol as a function of the eligibility rule and the collected patient data; and
determining a historic dynamic concordance rate for the protocol, the historic dynamic concordance rate for the protocol indicating the rate at which the patient data was consistent with the compliance rule for the protocol over the period of time.

2. The method as defined by claim 1, further comprising:
determining patient outcomes as a function of high compliance with the protocol relative to low compliance with the protocol.

3. The method as defined by claim 1, further comprising:
adjusting the eligibility rule or compliance rule for the protocol to define an adjusted protocol;
determining patient outcomes as a function of high compliance with the protocol relative to low compliance with the adjusted protocol.

4. The method of claim 3, wherein the collected patient data includes patient data from a newly deployed medical device,
wherein adjusting the protocol is a function of the patient data from the newly deployed medical device.

5. The method as defined by claim 1, further comprising:
receiving notification rule associated with the protocol;
determining a historic number of notifications over the period of time as a function of the notification rule;
adjusting the notification rule to reduce the historic number of notifications.

6. The method as defined by claim 1, wherein the protocol eligibility rule and compliance rule are generated using the graphic compiler.

7. The method as defined by claim 6, further comprising:
adjusting the protocol eligibility rule and/or compliance rule using the graphical compiler after receiving feedback regarding patient outcomes as a function of high compliance with the protocol, and/or
adjusting the protocol eligibility rule and/or compliance rule using the graphical compiler after receiving feedback regarding a number of notifications generated by the protocol.

8. A computer program product for use on a computer system for enrolling a patient in a medical protocol, the computer program product comprising a tangible, non-transient computer usable medium having computer readable program code thereon, the computer readable program code comprising:
program code for receiving eligibility rule and compliance rule for a medical protocol;
program code for receiving collected patient data from a plurality of patients, the collected patient data including longitudinal patient data relating to a specific parameter collected over a period of time from one or more sensors and/or medical devices;
program code for receiving determining when a patient was eligible for the protocol as a function of the eligibility rule and the collected patient data; and
program code for receiving determining a historic dynamic concordance rate for the protocol, the historic dynamic concordance rate for the protocol indicating the rate at which the patient data was consistent with the compliance rule for the protocol over the period of time.

9. The computer program product of claim 8, further comprising:
program code for determining patient outcomes as a function of high compliance with the protocol relative to low compliance with the protocol.

10. The computer program product of claim 8, further comprising:
program code for adjusting the eligibility rule or compliance rule for the protocol to define an adjusted protocol;
program code for determining patient outcomes as a function of high compliance with the protocol relative to low compliance with the adjusted protocol.

11. The computer program product of claim 8, further comprising:
program code for receiving notification rule associated with the protocol;
program code for determining a historic number of notifications over the period of time as a function of the notification rule;
program code for adjusting the notification rule to reduce the historic number of notifications.

12. The computer program product of claim 8, wherein the protocol eligibility rule and compliance rule are generated using the graphic compiler.

13. The computer program product of claim 12, further comprising:
program code for adjusting the protocol eligibility rule and/or compliance rule using the graphical compiler after receiving feedback regarding patient outcomes as a function of high compliance with the protocol.

14. The computer program product of claim 12, further comprising:
program code for adjusting the protocol eligibility rule and/or compliance rule using the graphical compiler after receiving feedback regarding a number of notifications generated by the protocol.

15. A system for simulating a medical protocol, the system comprising:
an application simulator configured to receive eligibility rule and compliance rule for a medical protocol, the simulator further configured to receive collected patient data from a plurality of patients, the collected patient data including longitudinal patient data relating to a specific parameter collected over a period of time from one or more sensors and/or medical devices,
the simulator further configured to compare the patient data with the eligibility rule to determine when a patient was eligible for the protocol, and
the simulator further configured to compare the patient data with the compliance rule to determine a historic dynamic concordance rate for the protocol, the historic dynamic concordance rate for the protocol indicating the rate at which the patient data was consistent with the compliance rule for the protocol over the period of time.

16. The system as defined by claim 15, further comprising a database including the eligibility rule and the compliance rule.

17. The system as defined by claim 16, wherein the application simulator is configured to receive notification rule associated with the protocol, and to determine the number of notifications as a function of the protocol and the patient data.

18. The system as defined by claim 16, further comprising a reporting module configured to provide a report about the protocol, wherein the report includes clicks by medical staff in the interface, interactions with the interface by medical staff, average patient compliance, time from eligibility to protocol start, ICU length of stay, ventilation time, extubation failure rate, readmission rate, and/or patient outcomes relative to dynamic concordance of the patient.

19. The system as defined by claim 18, further comprising a graphic compiler configured to graphically generate the eligibility rule and/or the compliance rule.

20. The system as defined by claim 17, wherein a rule of the protocol is modified using the graphical compiler after generating the report.
